(19) United States
(12) Patent Application Publication
(10) Pub. No.: US 2023/0145042 A1
(43) Pub. Date: May 11, 2023

(54) SYSTEMS AND METHODS FOR GENERATING AND DISPLAYING AN IMPLEMENTABLE TREATMENT PLAN BASED ON 2D INPUT IMAGES

(71) Applicant: SDC U.S. SmilePay SPV, Nashville, TN (US)

(72) Inventors: SERGEY NIKOLSKIY, Nashville, TN (US); RYAN AMELON, Coralville, IA (US); ANTON SERGEEVICH ZADORA, Moscow (RU); STANISLAV DMITRIEVICH GROKHOLSKII, Moscow (RU); TIM WUCHER, Windhoek (NA); JORDAN KATZMAN, Nashville, TN (US)

(73) Assignee: SDC U.S. SmilePay SPV, Nashville, TN (US)

(21) Appl. No.: 17/883,354

(22) Filed: Aug. 8, 2022

Related U.S. Application Data
(63) Continuation of application No. PCT/RU2021/000513, filed on Nov. 7, 2021.

(51) Int. Cl. A61C 7/00; A61C 13/34; G06F 3/01; G16H 20/40; G06Q 10/10

(52) U.S. Cl. CPC A61C 7/002; A61C 13/34; G06F 3/017; G16H 20/40; G06Q 10/1095

(57) ABSTRACT

A method of visualizing a treatment of teeth includes maintaining a final position model trained to output movement of teeth from initial to final positions, receiving from a user device a first representation comprising teeth of a user in an initial position where the first representation is based on a 2D image transmitted from the user device, determining tooth movements of the teeth from the initial position to a final position using to the final position model, receiving by the user device a graphical visualization of a treatment plan for moving the teeth of the user where the graphical visualization is generated based on the final position and the treatment plan is suitable for correcting the user's malocclusion, and displaying by the user device the graphical visualization where the graphical visualization comprises a three-dimensional (3D) representation corresponding to the teeth of the user.

### CROSS-REFERENCE TO RELATED APPLICATION

This application is a continuation of International Application No. PCT/RU2021/000513, filed Nov. 17, 2021, which is incorporated herein by reference in its entirety.

### TECHNICAL FIELD

The present disclosure relates generally to the field of dental treatment, and more specifically to systems and methods for automatically generating three dimensional (3D) teeth positions learned from full 3D teeth geometries, which are used for generating a treatment plan for orthodontic care.

### BACKGROUND

Some patients may receive treatment for misalignment of teeth using dental aligners. To provide the patient with dental aligners to treat the misalignment, a treatment plan is typically generated and/or approved by a treating dentist. The treatment plan may include 3D representations of the patient's teeth as they are expected to progress from their pre-treatment position (e.g., an initial position) to a target, final position selected by a treating dentist, taking into account a variety of clinical, practical and aesthetic factors. Selecting the final position typically involves an arduous process of selecting and moving teeth on an individual basis. Additionally, since the selection is made by a treating dentist, the final position determined or selected by the treating dentist typically involves the dentist's subjective opinion on the best treatment outcome.

### SUMMARY

In one aspect, this disclosure is directed to a method. The method includes maintaining, by one or more processors, a final position model trained to output movement of teeth of a dentition from initial positions to final positions given a plurality of compressed three-dimensional (3D) representations of dentitions comprising a plurality of teeth, maintaining, by the one or more processors, a geometric encoder model trained to compress a plurality of 3D representations of detentions comprising a plurality of teeth, receiving, by the one or more processors, a first 3D representation of a dentition comprising a plurality of teeth of a patient in an initial position where the first 3D representation comprises a plurality of tooth representations, generating, by the one or more processors, for each tooth representation, a compressed tooth representation using the geometric encoder model, determining, by the one or more processors, tooth movements of the plurality of teeth of the dentition from the initial position to a final position based on applying each compressed tooth representation to the final position model, and generating, by the one or more processors, a second 3D representation of the dentition comprising the plurality of teeth of the patient in the final position. Generating the second 3D representation comprises applying, by the one or more processors, the tooth movements to the first 3D representation of the detention comprising the plurality of teeth of the patient in the initial position.

In another aspect, this disclosure is directed to a treatment planning system. The treatment planning system includes one or more processors. The treatment planning system includes memory storing instructions that, when executed by the one or more processors, cause the one or more processors to maintain a final position model trained to output movement of teeth of a dentition from initial positions to final positions given a plurality of compressed three-dimensional (3D) representations of dentitions comprising a plurality of teeth, maintain a geometric encoder model trained to compress a plurality of 3D representations of detentions comprising a plurality of teeth, receive a first 3D representation of a dentition comprising a plurality of teeth of a patient in an initial position where the first 3D representation comprises a plurality of tooth representations, generate, for each tooth representation, a compressed tooth representation using the geometric encoder model, determine tooth movements of the plurality of teeth of the dentition from the initial position to a final position based on applying each compressed tooth representation to the final position model, and generate a second 3D representation of the dentition comprising the plurality of teeth of the patient in the final position. Generating the second 3D representation comprises applying the tooth movements to the first 3D representation of the detention comprising the plurality of teeth of the patient in the initial position

In another aspect, this disclosure is directed to a non-transitory computer readable medium storing instructions that, when executed by one or more processors, cause the one or more processors to maintain a final position model trained to output movement of teeth of a dentition from initial positions to final positions given a plurality of compressed three-dimensional (3D) representations of dentitions comprising a plurality of teeth, maintain a geometric encoder model trained to compress a plurality of 3D representations of detentions comprising a plurality of teeth, receive a first 3D representation of a dentition comprising a plurality of teeth of a patient in an initial position, the first 3D representation comprising a plurality of tooth representations, generate, for each tooth representation, a compressed tooth representation using the geometric encoder model, determine tooth movements of the plurality of teeth of the dentition from the initial position to a final position based on applying each compressed tooth representation to the final position model, and generate a second 3D representation of the dentition comprising the plurality of teeth of the patient in the final position. Generating the second 3D representation comprises applying the tooth movements to the first 3D representation of the detention comprising the plurality of teeth of the patient in the initial position

Various other embodiments and aspects of the disclosure will become apparent based on the drawings and detailed description of the following disclosure.

### BRIEF DESCRIPTION OF THE DRAWINGS

FIG. 1 shows a system for orthodontic treatment, according to an illustrative embodiment.

FIG. 2 shows a process flow of generating a treatment plan, according to an illustrative embodiment.

FIG. 3 shows a top-down simplified view of a model of a dentition, according to an illustrative embodiment.

FIG. 4 shows a perspective view of a three-dimensional model of the dentition of FIG. 3, according to an illustrative embodiment.

FIG. 5 shows a trace of a gingiva-tooth interface on the model shown in FIG. 3, according to an illustrative embodiment.

FIG. 6 shows selection of teeth in a tooth model generated from the model shown in FIG. 5, according to an illustrative embodiment.

FIG. 7 shows a segmented tooth model of an initial position of the dentition shown in FIG. 3, according to an illustrative embodiment.

FIG. 8 shows a target final position of the dentition from the initial position of the dentition shown in FIG. 7, according to an illustrative embodiment.

FIG. 9 shows a series of stages of the dentition from the initial position shown in FIG. 7 to the target final position shown in FIG. 8, according to an illustrative embodiment.

FIG. 10 shows a view of a final position processing engine of a treatment planning computing system of FIG. 1, according to an illustrative embodiment.

FIG. 11 shows a block diagram of an example system using supervised learning that may be used to determine a final position of the teeth, according to an illustrative embodiment.

FIG. 12 shows a block diagram of a simplified neural network model, according to an illustrative embodiment.

FIGS. 13A-13D show a system for generating a treatment plan, according to illustrative embodiments.

FIG. 14 is a flowchart showing a method of automatically determining a final position of a patient's dentition, according to an illustrative embodiment.

FIGS. 15A-15B show examples of a user approving a treatment plan, according to illustrative embodiments.

FIG. 16 is a flowchart showing a method of automatically verifying the safety and clinical efficiency of an orthodontic treatment plan by performing a clinical assessment on the treatment plan

### DETAILED DESCRIPTION

The present disclosure is directed to systems and methods for automatically determining a final treatment plan position of a patient's dentition. According to various embodiments, the systems and methods described herein may include maintaining a geometric encoder model and a final position model. The final position model may be configured to determine movement of teeth (including translation movement and rotation movement) of a dentition from initial positions to final treatment planning positions. The final position model may be trained on a training set comprising a plurality of compressed three-dimensional (3D) training representations of dentitions comprising a plurality of teeth, and corresponding tooth movements to respective planned final positions post-treatment. The geometric encoder model may be configured to encode a 3D geometry representing surfaces of teeth into compressed representations. The geometric encoder models may be part of an autoencoder trained to encode 3D geometric information (such as point cloud distributions) into a compressed representation (such as a vector of floating point values). The systems and methods described herein may receive a first 3D representation of a dentition comprising a plurality of teeth of a patient in an initial position. The first 3D representation may include a plurality of tooth representations including a plurality of points representing surfaces of a respective tooth of the dentition. The systems and methods described herein may generate, for each tooth representation, a compressed tooth representation by transforming the initial 3D representation using the geometric encoder model. The systems and methods described herein may determine tooth movements of the plurality of teeth of the dentition from the initial position to a final position, responsive to applying each compressed tooth representation to the final position model. The systems and methods described herein may generate a second 3D representation of the dentition comprising the plurality of teeth of the patient in the final treatment planning position. Generating the second 3D representation may include applying the tooth movements to the initial 3D representation to move the orientation and rotation of each tooth (or group of teeth) into a final treatment plan position.

The systems and methods described herein may be trained based on previous (or historic) treatment plan data. The treatment plan data may be maintained or stored by a provider of the dental aligners. In some embodiments, the previous treatment plan data may be limited to treatment plans deemed successful (e.g., treatment plans which did not require a mid-course correction, treatment plans receiving positive patient feedback in a survey, etc.). The previous treatment plan data may include, for example, 3D data corresponding to an initial position of the previous patient's dentition, teeth movement data (e.g., translation and/or rotation movements from the initial position to a respective final position), and/or 3D data corresponding to the final position of the previous patient's dentition, etc.

By using previous or historic treatment plan data to train the machine learning models set forth herein, the systems and methods described herein may learn to leverage full and actual 3D geometries for training or learning treatment planning movements. Other solutions may involve identifying previous similar cases in a database or other data structure, which can be time/resource consuming to identify, and may be problematic where a similar case has not been treated before. Rather, by training based on previous or historic treatment plan data, the systems and methods described herein may be capable of identifying or learning teeth movements for any combination of teeth positions, which results in a more flexible final position model. Additionally, since the systems and methods herein rely on full and actual 3D geometries for training or learning treatment planning movements rather than hand-crafted geometric information (such as landmarks performed by a human as part of labeling or training), the systems and methods described herein may not discard important 3D data used for training. For example, where landmarking is performed in other solutions, such solutions may not be trained on full 3D data sets. Since the full 3D data sets are not used, the treatment plans generated from such solutions may be based on incomplete data. On the other hand, by relying on full 3D data sets as described herein, the systems and methods described herein may be trained on more complete data and therefore result in more accurate treatment plans.

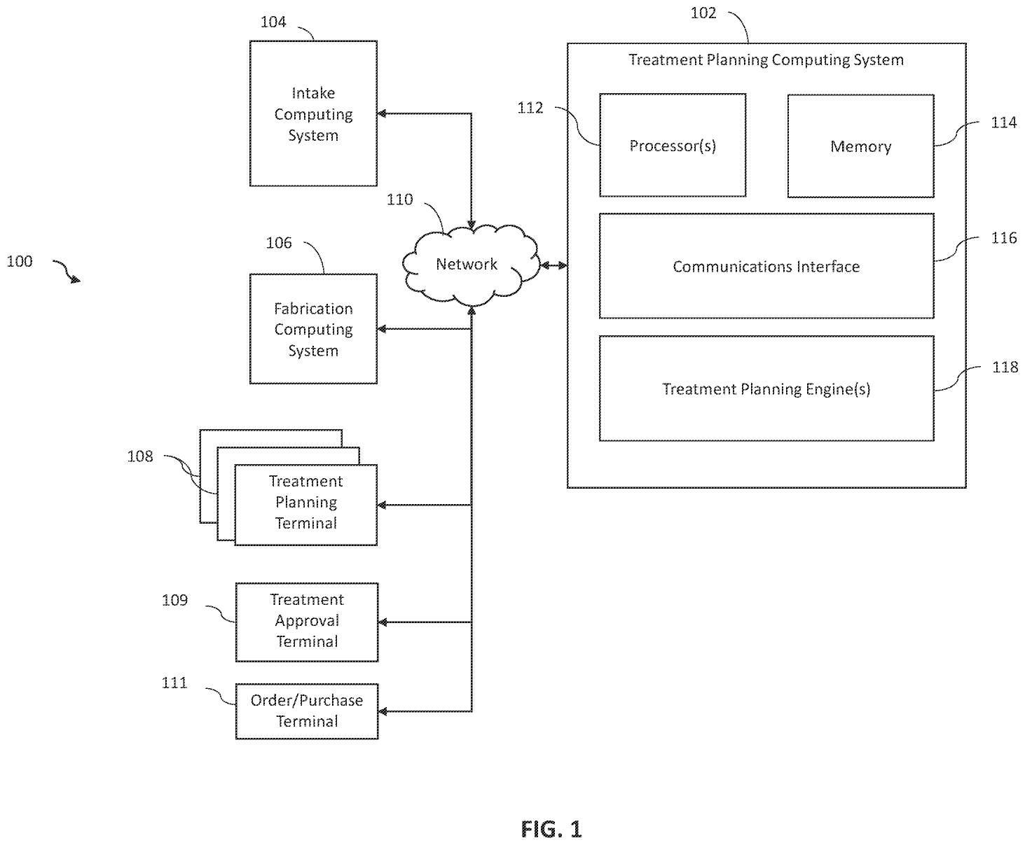

Referring to FIG. 1, a system 100 for orthodontic treatment is shown, according to an illustrative embodiment. As shown in FIG. 1, the system 100 includes a treatment planning computing system 102 communicably coupled to an intake computing system 104, a fabrication computing system 106, and one or more treatment planning terminals 108. In some embodiments, the treatment planning computing system 102 may be or may include one or more servers which are communicably coupled to a plurality of computing devices. In some embodiments, the treatment planning computing system 102 may include a plurality of servers, which may be located at a common location (e.g., a server bank) or may be distributed across a plurality of locations. The treatment planning computing system 102 may be communicably coupled to the intake computing system 104, fabrication computing system 106, treatment approval terminal 109, order/purchase terminal 111, and/or treatment planning terminals 108 via a communications link or network 110 (which may be or include various network connections configured to communicate, transmit, receive, or otherwise exchange data between addresses corresponding to the computing systems 102, 104, 106, 109, 111). The network 110 may be a Local Area Network (LAN), a Wide Area Network (WAN), a Wireless Local Area Network (WLAN), an Internet Area Network (IAN) or cloud-based network, etc. The network 110 may facilitate communication between the respective components of the system 100, as described in greater detail below.

The computing systems 102, 104, 106, 109, 111 include one or more processing circuits, which may include processor(s) 112 and memory 114. The processor(s) 112 may be a general purpose or specific purpose processor, an application specific integrated circuit (ASIC), one or more field programmable gate arrays (FPGAs), a group of processing components, or other suitable processing components. The processor(s) 112 may be configured to execute computer code or instructions stored in memory 114 or received from other computer readable media (e.g., CDROM, network storage, a remote server, etc.) to perform one or more of the processes described herein. The memory 114 may include one or more data storage devices (e.g., memory units, memory devices, computer-readable storage media, etc.) configured to store data, computer code, executable instructions, or other forms of computer-readable information. The memory 114 may include random access memory (RAM), read-only memory (ROM), hard drive storage, temporary storage, non-volatile memory, flash memory, optical memory, or any other suitable memory for storing software objects and/or computer instructions. The memory 114 may include database components, object code components, script components, or any other type of information structure for supporting the various activities and information structures described in the present disclosure. The memory 114 may be communicably connected to the processor 112 via the processing circuit, and may include computer code for executing (e.g., by processor(s) 112) one or more of the processes described herein.

The order/purchase terminal 111 may include any device(s), component(s), circuit(s), or other combination of hardware components designed or implemented to complete and/or guide a user in placing an order. An order may be a transaction that exchanges money from a patient for a product (e.g., an impression kit, dental aligners, etc.). The order/purchase terminal 111 may communicate with the fabrication computing system 106 and third party device (e.g., a patient or other user device) to guide a patient or other user through a payment/order completion system. In some embodiments, the order/purchase terminal 111 may communicate prompts to the user device to guide the user through the payment/order completion system. The prompts may include asking the patient for patient information (e.g., name, physical address, email address, phone number, credit card information) and product information (e.g., quantity of product, product name). In response to receiving information from the patient, the order/purchase terminal 111 initiates a product order. The initiated product order is transmitted to the fabrication computing system 106 to initiate the fabrication of one or more products (e.g., dental aligners). The initiated product order may also be transmitted to the intake computing system 104 to store/record the transaction and/or to initiate a product order from the computing system (e.g., a dental impression kit), and the like.

The treatment planning computing system 102 is shown to include a communications interface 116. The communications interface 116 can be or can include components configured to transmit and/or receive data from one or more remote sources (such as the computing devices, components, systems, and/or terminals described herein). In some embodiments, each of the servers, systems, terminals, and/or computing devices may include a respective communications interface 116 which permit exchange of data between the respective components of the system 100. As such, each of the respective communications interfaces 116 may permit or otherwise enable data to be exchanged between the respective computing systems 102, 104, 106, 109, 111. In some implementations, communications device(s) may access the network 110 to exchange data with various other communications device(s) via cellular access, a modem, broadband, Wi-Fi, satellite access, etc. via the communications interfaces 116.

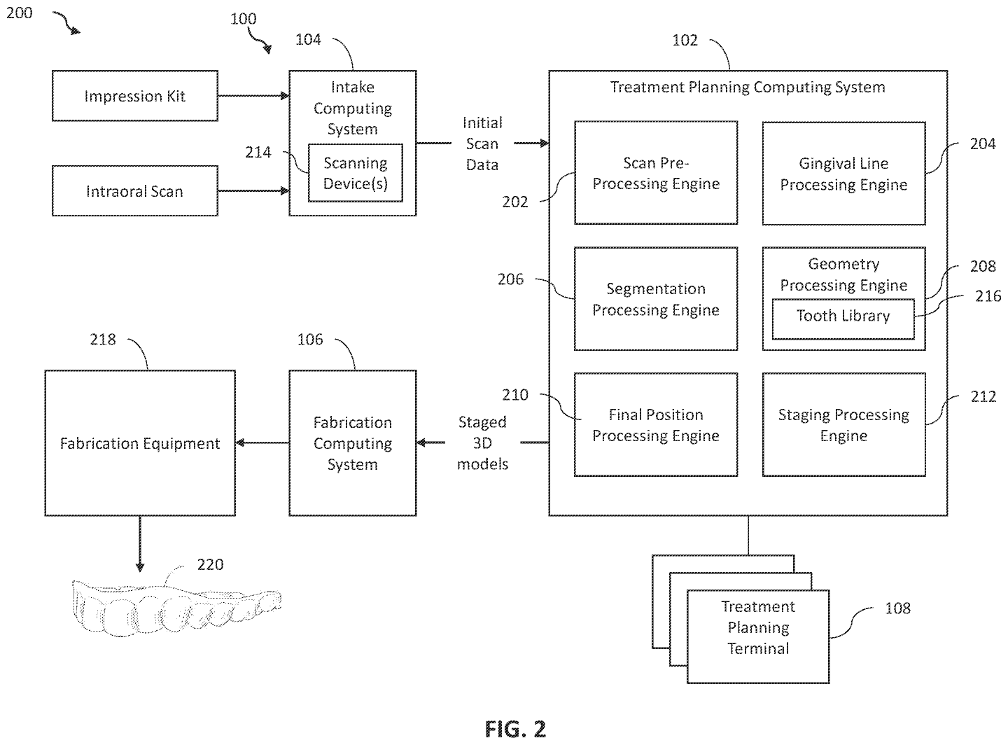

Referring now to FIG. 1 and FIG. 2, the treatment planning computing system 102 is shown to include one or more treatment planning engines 118. Specifically, FIG. 2 shows a treatment planning process flow 200 which may be implemented by the system 100 shown in FIG. 2, according to an illustrative embodiment. The treatment planning engine(s) 118 may be any device(s), component(s), circuit(s), or other combination of hardware components designed or implemented to receive inputs for and/or automatically generate a treatment plan from an initial three-dimensional (3D) model of a dentition. In some embodiments, the treatment planning engine(s) 118 may be instructions stored in memory 114 which are executable by the processor(s) 112. In some embodiments, the treatment planning engine(s) 118 may be stored at the treatment planning computing system 102 and accessible via a respective treatment planning terminal 108. As shown in FIG. 2, the treatment planning computing system 102 may include a scan pre-processing engine 202, a gingival line processing engine 204, a segmentation processing engine 206, a geometry processing engine 208, a final position processing engine 210, and a staging processing engine 212. While these engines 202-212 are shown in FIG. 2, it is noted that the system 100 may include any number of treatment planning engines 118, including additional engines which may be incorporated into, supplement, or replace one or more of the engines shown in FIG. 2.

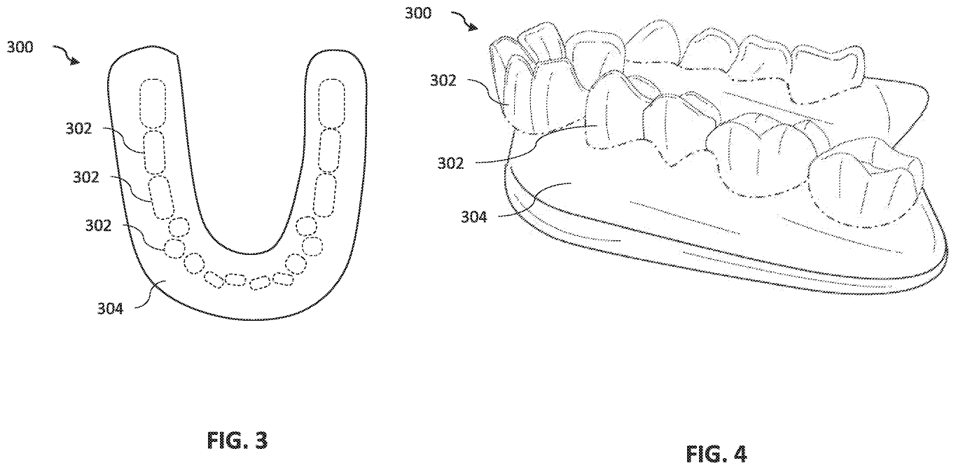

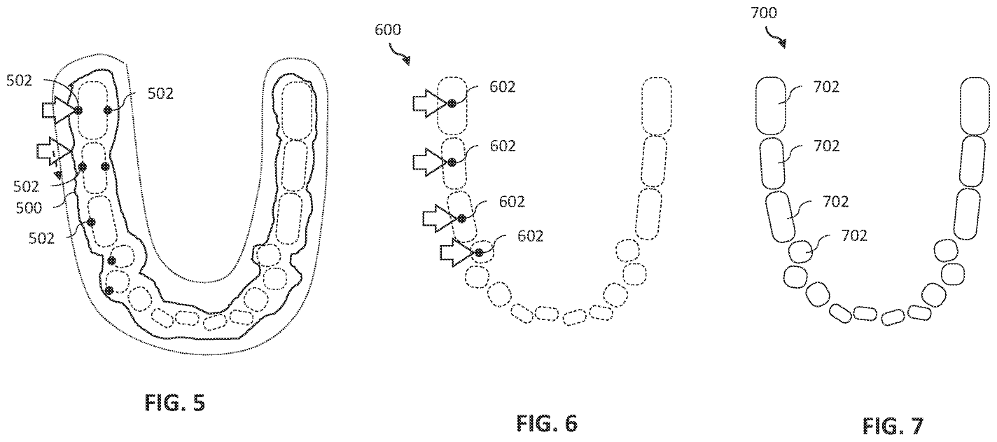

Referring to FIG. 2-FIG. 4, the intake computing system 104 may be configured to generate a 3D model of a dentition. Specifically, FIG. 3 and FIG. 4 show a simplified top-down view and a side perspective view of a 3D model of a dentition, respectively, according to illustrative embodiments. In some embodiments, the intake computing system 104 may be communicably coupled to or otherwise include one or more scanning devices 214. The intake computing system 104 may be communicably coupled to the scanning devices 214 via a wired or wireless connection. The scanning devices 214 may be or include any device, component, or hardware designed or implemented to generate, capture, or otherwise produce a 3D model 300 of an object, such as a dentition or dental arch. In some embodiments, the scanning devices 214 may include intraoral scanners configured to generate a 3D model of a dentition of a patient as the intraoral scanner passes over the dentition of the patient. For example, the intraoral scanner may be used during an intraoral scanning appointment, such as the intraoral scanning appointments described in U.S. Provisional Patent Appl. No. 62/660,141, titled “Arrangements for Intraoral Scanning,” filed Apr. 19, 2018, and U.S. patent application Ser. No. 16/130,762, titled “Arrangements for Intraoral Scanning,” filed Sep. 13, 2018. In some embodiments, the scanning devices 214 may include 3D scanners configured to scan a dental impression. The dental impression may be captured or administered by a patient using a dental impression kit similar to the dental impression kits described in U.S. Patent Application No. U.S. Provisional Patent Appl. No. 62/522,847, titled “Dental Impression Kit and Methods Therefor,” filed Jun. 21, 2017, and U.S. patent application Ser. No. 16/047,694, titled “Dental Impression Kit and Methods Therefor,” filed Jul. 27, 2018, the contents of each of which are incorporated herein by reference in their entirety. In these and other embodiments, the scanning device(s) 214 may generally be configured to generate a 3D digital model of a dentition of a patient. As an example, the 3D digital model may be a point cloud representation of the dentition, a voxel representation, a spline representation, a mesh representation, or any other parametric model representation. In some embodiments, the scanning device(s) 214 may be configured to capture a two dimensional (2D) image of a dentition of the patient. The scanning device(s) 214 may be configured to generate a 3D digital model of the upper (i.e., maxillary) dentition and/or the lower (i.e., mandibular) dentition of the patient. The 3D digital model may include a digital representation of the patient's teeth 302 and/or gingiva 304. The scanning device(s) 214 may be configured to generate 3D digital models of the patient's dentition prior to treatment (i.e., with their teeth in an initial position). In some embodiments, the scanning device(s) 214 may be configured to generate the 3D digital models of the patient's dentition in real-time (e.g., as the dentition/impression) is scanned. In some embodiments, the scanning device(s) 214 may be configured to export, transmit, send, or otherwise provide data obtained during the scan to an external source which generates the 3D digital model, and transmits the 3D digital model to the intake computing system 104.

The intake computing system 104 may be configured to transmit, send, or otherwise provide the 3D digital model to the treatment planning computing system 102. In some embodiments, the intake computing system 104 may be configured to provide the 3D digital model of the patient's dentition to the treatment planning computing system 102 by uploading the 3D digital model to a patient file for the patient. The intake computing system 104 may be configured to provide the 3D digital model of the patient's upper and/or lower dentition at their initial (i.e., pre-treatment) position. The 3D digital model of the patient's upper and/or lower dentition may together form initial scan data which represents an initial position of the patient's teeth prior to treatment.

The treatment planning computing system 102 may be configured to receive the initial scan data from the intake computing system 104 (e.g., from the scanning device(s) 214 directly, indirectly via an external source following the scanning device(s) 214 providing data captured during the scan to the external source, etc.). As described in greater detail below, the treatment planning computing system 102 may include one or more treatment planning engines 118 configured or designed to generate a treatment plan based on or using the initial scan data.

Referring to FIG. 2, the treatment planning computing system 102 is shown to include a scan pre-processing engine 202. The scan pre-processing engine 202 may be or include any device(s), component(s), circuit(s), or other combination of hardware components designed or implemented to modify, correct, adjust, or otherwise process initial scan data received from the intake computing system 104 prior to generating a treatment plan. Generally, the scan pre-processing engine 202 may be configured to standardize and/or normalize the initial scan data such that subsequent processing (e.g., the final position processing engine) operates (and learns) on stable data (e.g., data that is not significantly varied with respect to number of points in a point cloud or other 3D representation, noise, smoothing artifacts, and the like). In some implementations, if a scan pre-processing engine 202 is employed to modify the initial scan data, a post-processing engine (not shown) may be employed to modify, correct, adjust, or otherwise process the output data. For example, if an input point cloud is upsampled during pre-processing, then the output point cloud may be downsampled during post-processing. The scan pre-processing engine 202 may be configured to process the initial scan data by applying one or more surface smoothing, resampling, and/or artifact removing algorithms to the initial scan data and/or 3D digital models. The scan pre-processing engine 202 may be configured to fill one or more holes or gaps in the 3D digital models. In some embodiments, the scan pre-processing engine 202 may be configured to receive inputs from a treatment planning terminal 108 to process the initial scan data. For example, the scan pre-processing engine 202 may be configured to receive inputs to smooth, refine, adjust, or otherwise process the initial scan data.

The inputs may include a selection of a smoothing processing tool presented on a user interface of the treatment planning terminal 108 showing the 3D digital model(s). As a user of the treatment planning terminal 108 selects various portions of the 3D digital model(s) using the smoothing processing tool, the scan pre-processing engine 202 may correspondingly smooth the 3D digital model at (and/or around) the selected portion. Similarly, the scan pre-processing engine 202 may be configured receive a selection of a gap filling processing tool presented on the user interface of the treatment planning terminal 108 to fill gaps in the 3D digital model(s).

In some embodiments, the scan pre-processing engine 202 may be configured to receive inputs for removing a portion of the gingiva represented in the 3D digital model of the dentition. For example, the scan pre-processing engine 202 may be configured to receive a selection (on a user interface of the treatment planning terminal 108) of a gingiva trimming tool which selectively removes gingival form the 3D digital model of the dentition. A user of the treatment planning terminal 108 may select a portion of the gingiva to remove using the gingiva trimming tool. The portion may be a lower portion of the gingiva represented in the digital model opposite the teeth. For example, where the 3D digital model shows a mandibular dentition, the portion of the gingiva removed from the 3D digital model may be the lower portion of the gingiva closest to the lower jaw. Similarly, where the 3D digital model shows a maxillary dentition, the portion of the gingiva removed from the 3D digital model may be the upper portion of the gingiva closest to the upper jaw.

The scan pre-processing engine 202 may also be configured to generate the 3D digital data given other representations of data (e.g., 2D images) and subsequently smooth/modify/trim the 3D digital data as described herein. For example, the 3D digital data may be obtained using 2D image reconstruction. In one embodiment, the scan pre-processing engine 202 may employ photogrammetry, for instance, to extract 3D measurements from captured 2D images (e.g., captured images from the scanning device 214). The scan pre-processing engine 202 may perform photogrammetry by comparing known measurements (e.g., known tooth measurements) with measurements of tooth features in the 2D image. The lengths/sizes of various features include tooth size measurements, tooth orientation measurements, and the like. Performing photogrammetry results in the determination of a position, orientation, size, and/or rotation of a tooth in the image. In some embodiments, the scan pre-processing engine 202 may perform photogrammetry using measurements of average teeth features from one or more databases (e.g., stored in memory 114). In some embodiments, the scan pre-processing engine 202 may receive particular measurements of a patient (e.g., entered into by a user at a treatment planning terminal 108 when the patient is present at the treatment planning terminal). The scan pre-processing engine 202 may compare the known measurements of teeth features with dimensions/measurements of the teeth features in the captured image to determine the position, orientation, size and/or rotation of the teeth features in the image.

Additionally or alternatively, the scan pre-processing engine 202 may use triangulation to generate a three-dimensional model of the patient based on images from various perspectives (e.g., multiple images captured). As an example, the scan pre-processing engine 202 may associate a two-dimensional pixel in the subsequent images with a ray in three-dimensional space. Given multiple perspectives of the image (e.g., at least two subsequent images capture the position of the patient from at least two different perspectives), the scan pre-processing engine 202 may determine a three-dimensional point from the intersection of at least two rays from pixels of the subsequent images.

The scan pre-processing engine 202 may execute various consistency functions to determine that the rays from the subsequent images are associated with consistent pixels. For instance, a pixel from a first perspective of an image, mapped to a three-dimensional point using a ray based on the first perspective of the image, is consistent with a pixel from a second perspective of an image mapped to the same three-dimensional point using a ray from the second perspective of the image. The scan pre-processing engine 202 may determine from the consistency functions, whether the pixels used to determine the three-dimensional point have similar colors, similar textures the textures, similar opacity, and the like.

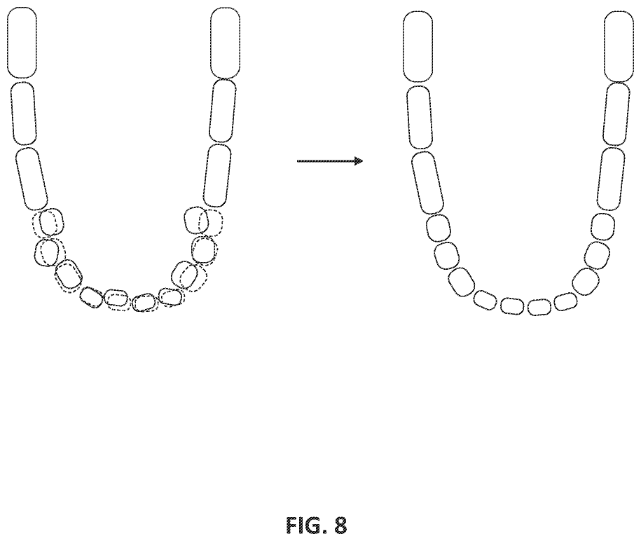

Referring now to FIG. 2 and FIG. 5, the treatment planning computing system 102 is shown to include a gingival line processing engine 204. Specifically, FIG. 5 shows a trace of a gingiva-tooth interface on the model 300 shown in FIG. 3 and FIG. 4. The gingival line processing engine 204 may be or include any device(s), component(s), circuit(s), or other combination of hardware components designed or implemented to determine, identify, or otherwise define a gingival line of the 3D digital models. The gingival line may be or include the interface between the gingiva and teeth represented in the 3D digital models. In some embodiments, the gingival line processing engine 204 may be configured to receive inputs from the treatment planning terminal 108 for defining the gingival line. The treatment planning terminal 108 may show a gingival line defining tool on a user interface which includes the 3D digital models.

The gingival line defining tool may be used for defining or otherwise determining the gingival line for the 3D digital models. As one example, the gingival line defining tool may be used to trace a rough gingival line 500. For example, a user of the treatment planning terminal 108 may select the gingival line defining tool on the user interface, and drag the gingival line defining tool along an approximate gingival line of the 3D digital model. As another example, the gingival line defining tool may be used to select (e.g., on the user interface shown on the treatment planning terminal 108) lowest points 502 at the teeth-gingiva interface for each of the teeth in the 3D digital model.

The gingival line processing engine 204 may be configured to receive the inputs provided by the user via the gingival line defining tool on the user interface of the treatment planning terminal 108 for generating or otherwise defining the gingival line. In some embodiments, the gingival line processing engine 204 may be configured to use the inputs to identify a surface transition on or near the selected inputs. For example, where the input selects a lowest point 502 (or a portion of the rough gingival line 500 near the lowest point 502) on a respective tooth, the gingival line processing engine 204 may identify a surface transition or seam at or near the lowest point 502 which is at the gingival margin. The gingival line processing engine 204 may define the transition or seam as the gingival line. In some embodiments, the gingival line processing engine 204 may automatically determine the gingival line (e.g., without receiving inputs from the treatment planning terminal 108) by segmenting each tooth (a portion of teeth, a group of teeth) to determine the teeth-gingiva interface. Accordingly, the gingival line processing engine 204 may be configured to differentiate teeth and gingiva in the 3D digital model via one or more image processing algorithms and/or machine learning algorithms trained to differentiate teeth from gingiva). The gingival line processing engine 204 may define the gingival line for each of the teeth 302 (or a portion/group of teeth) included in the 3D digital model 300 (or a 2D image). The gingival line processing engine 204 may be configured to generate a tooth model using the gingival line of the teeth 302 in the 3D digital model 300. The gingival line processing engine 204 may be configured to generate the tooth model by separating the 3D digital model along the gingival line. The tooth model may be the portion of the 3D digital model which is separated along the gingival line and includes digital representations of the patient's teeth.

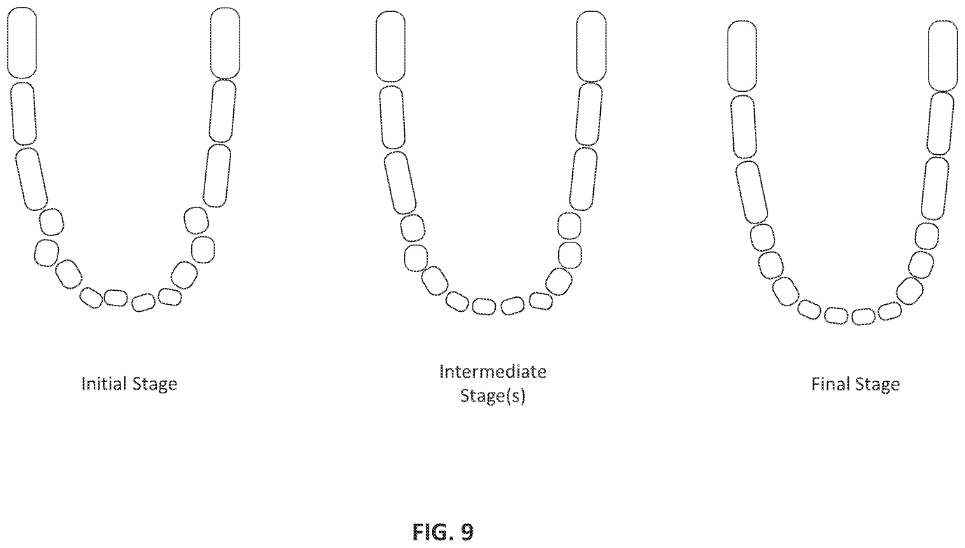

Referring now to FIG. 2 and FIG. 6, the treatment planning computing system 102 is shown to include a segmentation processing engine 206. Specifically, FIG. 6 shows a view of the tooth model 600 generated by the gingival line processing engine 204. The segmentation processing engine 206 may be or include any device(s), component(s), circuit(s), or other combination of hardware components designed or implemented to determine, identify, or otherwise segment individual teeth from the tooth model. For example, the segmentation processing engine 206 may be configured to differentiate teeth in the tooth model 600 via one or more image processing algorithms and/or machine learning algorithms trained to differentiate teeth. In some embodiments, the segmentation processing engine 206 may be configured to receive inputs (e.g., via a user interface shown on the treatment planning terminal 108) which select the teeth (e.g., points 602 on the teeth) in the tooth model 600. For example, the user interface may include a segmentation tool which, when selected, allows a user to select points 602 on each of the individual teeth in the tooth model 600. In some embodiments, the selection of each teeth may also assign a label to the teeth. The label may include tooth numbers (e.g., according to FDI world dental federation notation, the universal numbering system, Palmer notation, etc.) for each of the teeth in the tooth model 600. As shown in FIG. 6, the user may select individual teeth in the tooth model 600 to assign a label to the teeth.

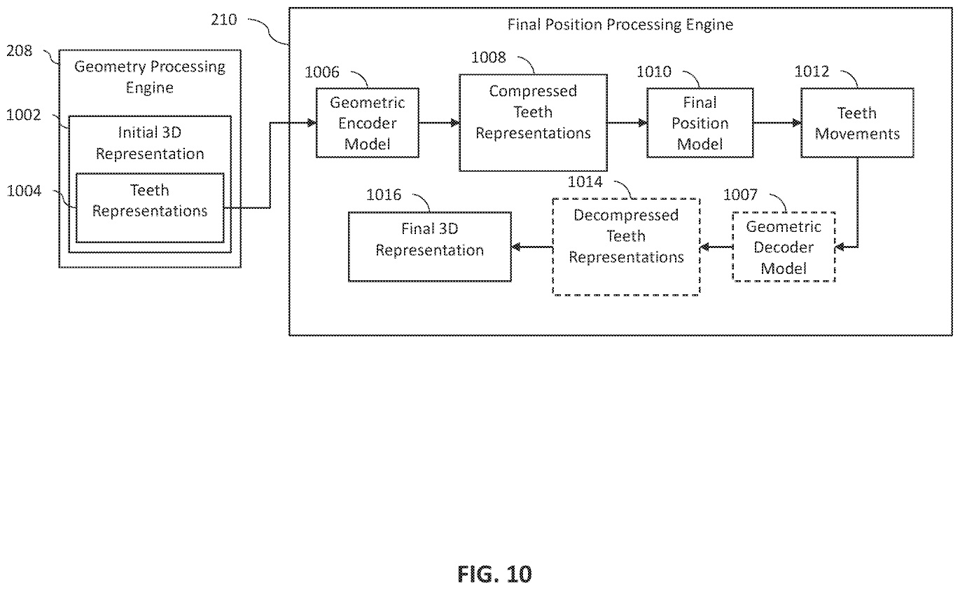

Referring now to FIG. 7, depicted is a segmented tooth model 700 generated from the tooth model 600 shown in FIG. 6. The segmentation processing engine 206 may be configured to receive the selection of the teeth from the user via the user interface of the treatment planning terminal 108. The segmentation processing engine 206 may be configured to separate each of the teeth selected by the user on the user interface. For example, the segmentation processing engine 206 may be configured to identify or determine a gap between two adjacent points 602. The segmentation processing engine 206 may be configured to use the gap as a boundary defining or separating two teeth. The segmentation processing engine 206 may be configured to define boundaries for each of the teeth in the tooth model 600. The segmentation processing engine 206 may be configured to generate the segmented tooth model 700 including segmented teeth 702 using the defined boundaries generated from the selection of the points 602 on the teeth in the tooth model 600.

The treatment planning computing system 102 is shown to include a geometry processing engine 208. The geometry processing engine 208 may be or include any device(s), component(s), circuit(s), or other combination of hardware components designed or implemented to determine, identify, or otherwise generate whole tooth models for each of the teeth in the 3D digital model. Once the segmentation processing engine 206 generates the segmented tooth model 700, the geometry processing engine 208 may be configured to use the segmented teeth to generate a whole tooth model for each of the segmented teeth. Since the teeth have been separated along the gingival line by the gingival line processing engine 204 (as described above with reference to FIG. 6), the segmented teeth may only include crowns (e.g., the segmented teeth may not include any roots). The gingival line processing engine 204 may be configured to generate a whole tooth model including both crown and roots using the segmented teeth. In some embodiments, the segmentation processing engine 206 may be configured to generate the whole tooth models using the labels assigned to each of the teeth in the segmented tooth model 700. For example, the geometry processing engine 208 may be configured to access a tooth library 216. The tooth library 216 may include a library or database having a plurality of whole tooth models. The plurality of whole tooth models may include tooth models for each of the types of teeth in a dentition. The plurality of whole tooth models may be labeled or grouped according to tooth numbers.

The geometry processing engine 208 may be configured to generate the whole tooth models for a segmented tooth by performing a look-up function in the tooth library 216 using the label assigned to the segmented tooth to identify a corresponding whole tooth model. The geometry processing engine 208 may be configured to morph the whole tooth model identified in the tooth library 216 to correspond to the shape (e.g., surface contours) of the segmented tooth. In some embodiments, the geometry processing engine 208 may be configured to generate the whole tooth model by stitching the morphed whole tooth model from the tooth library 216 to the segmented tooth, such that the whole tooth model includes a portion (e.g., a root portion) from the tooth library 216 and a portion (e.g., a crown portion) from the segmented tooth. In some embodiments, the geometry processing engine 208 may be configured to generate the whole tooth model by replacing the segmented tooth with the morphed tooth model from the tooth library. In these and other embodiments, the geometry processing engine 208 may be configured to generate whole tooth models, including both crown and roots, for each of the teeth in a 3D digital model. The whole tooth models of each of the teeth in the 3D digital model may depict, show, or otherwise represent an initial position of the patient's dentition.

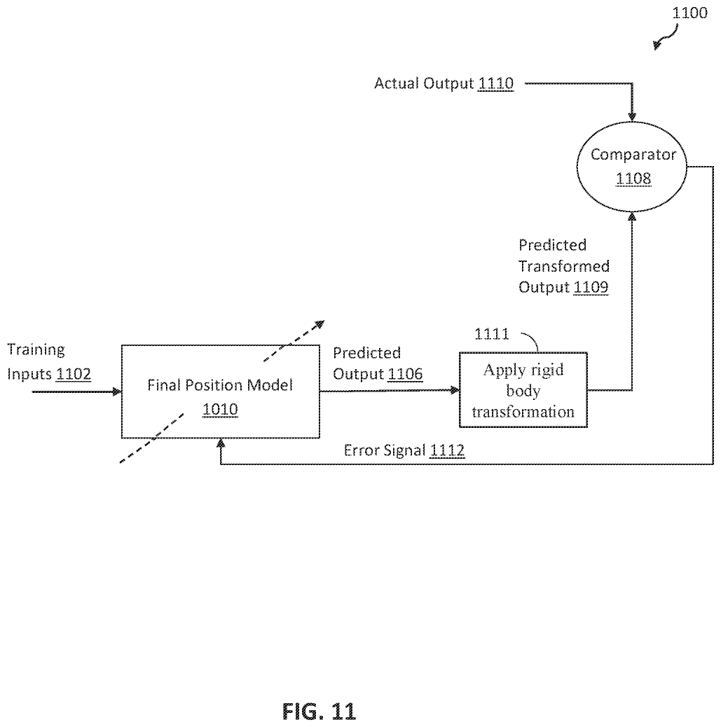

Referring now to FIG. 2 and FIG. 8, the treatment planning computing system 102 is shown to include a final position processing engine 210. FIG. 8 shows one example of a target final position of the dentition from the initial position of the dentition shown in FIG. 7 from a top-down view. The final position processing engine 210 may be or may include any device(s), component(s), circuit(s), or other combination of hardware components designed or implemented to determine, identify, or otherwise generate (or determine) a final position of the patient's teeth. The final position processing engine 210 may be configured to generate the treatment plan by manipulating individual 3D models of teeth within the 3D model (e.g., shown in FIG. 7). In some embodiments, the final position processing engine 210 may be configured to receive inputs for generating the final position of the patient's teeth. The final position may be a target position of the teeth post-orthodontic treatment or at a last stage of realignment. A user of the treatment planning terminal 108 may provide one or more inputs for each tooth or a subset of the teeth in the initial 3D model to move the teeth from their initial position to their final position (shown in dot-dash). For example, the treatment planning terminal 108 may be configured to receive inputs to drag, shift, rotate, or otherwise move individual teeth to their final position, incrementally shift the teeth to their final position, etc. The movements may include lateral/longitudinal movements, rotation movements, translational movements, etc. The movements may include intrusions and/or extrusions of the teeth relative to the occlusal axis, as will be described below.

In some embodiments, the manipulation of the 3D model may show a final (or target) position of the teeth of the patient following orthodontic treatment or at a last stage of realignment via dental aligners. In some embodiments, the final position processing engine 210 may be configured to apply one or more movement thresholds (e.g., a maximum lateral and/or rotation movement for treatment) to each of the individual 3D teeth models for generating the final position. As such, the final position may be generated in accordance with the movement thresholds.

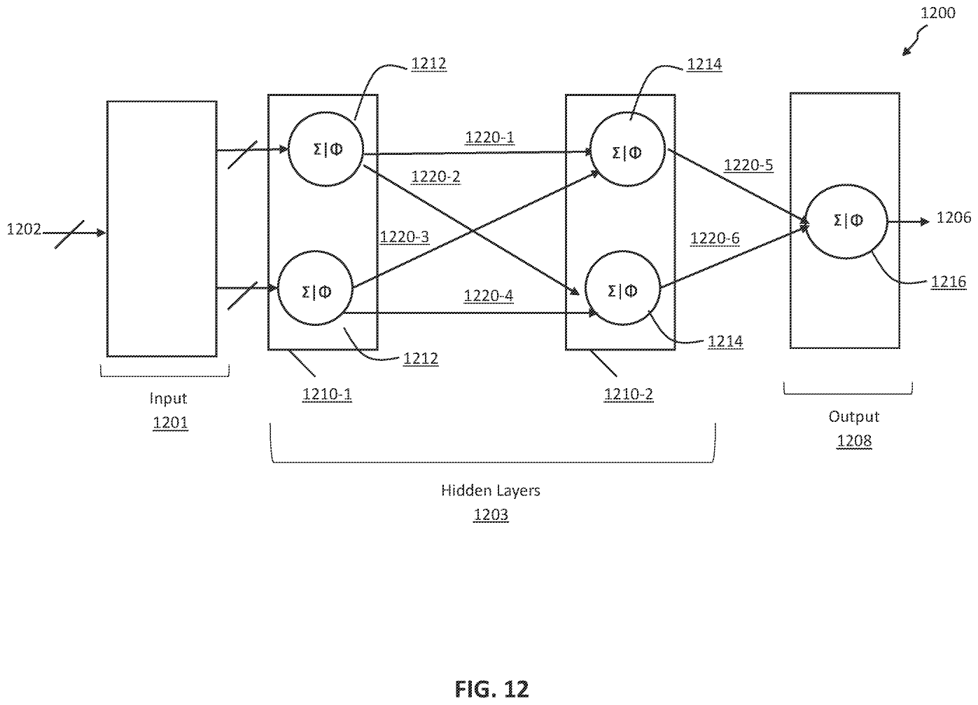

Referring now to FIG. 2 and FIG. 9, the treatment planning computing system 102 is shown to include a staging processing engine 212. Specifically, FIG. 9 shows a series of stages of the dentition from the initial position shown in FIG. 7 to the target final position shown in FIG. 8, according to an illustrative embodiment. The staging processing engine 212 may be or include any device(s), component(s), circuit(s), or other combination of hardware components designed or implemented to determine, identify, or otherwise generate stages of treatment (e.g., a treatment plan) from the initial position to the final position of the patient's teeth. In some embodiments, the staging processing engine 212 may be configured to receive inputs (e.g., via a user interface of the treatment planning terminal 108) for generating the stages. In some embodiments, the staging processing engine 212 may be configured to automatically compute or determine the stages based on the movements from the initial to the final position. The staging processing engine 212 may be configured to apply one or more movement thresholds (e.g., a maximum lateral and/or rotation movement for a respective stage) to each stage of treatment plan. The staging processing engine 212 may be configured to generate the stages as 3D digital models of the patient's teeth as they progress from their initial position to their final position. For example, and as shown in FIG. 9, the stages may include an initial stage including a 3D digital model of the patient's teeth at their initial position, one or more intermediate stages including 3D digital model(s) of the patient's teeth at one or more intermediate positions, and a final stage including a 3D digital model of the patient's teeth at the final position.

In some embodiments, the staging processing engine 212 may be configured to generate at least one intermediate stage for each tooth based on a difference between the initial position of the tooth and the final position of the tooth. For instance, where the staging processing engine 212 generates one intermediate stage, the intermediate stage may be a halfway point between the initial position of the tooth and the final position of the tooth. Each of the stages may together form a treatment plan for the patient, and may include a series or set of 3D digital models.

Following generating the stages, the treatment planning computing system 102 may be configured to transmit, send, or otherwise provide the staged 3D digital models to the fabrication computing system 106. In some embodiments, the treatment planning computing system 102 may be configured to provide the staged 3D digital models to the fabrication computing system 106 by uploading the staged 3D digital models to a patient file which is accessible via the fabrication computing system 106. In some embodiments, the treatment planning computing system 102 may be configured to provide the staged 3D digital models to the fabrication computing system 106 by sending the staged 3D digital models to an address (e.g., an email address, IP address, etc.) for the fabrication computing system 106.

The fabrication computing system 106 can include a fabrication computing device and fabrication equipment 218 configured to produce, manufacture, or otherwise fabricate dental aligners. The fabrication computing system 106 may be configured to receive a plurality of staged 3D digital models corresponding to the treatment plan for the patient. As stated above, each 3D digital model may be representative of a particular stage of the treatment plan (e.g., a first 3D model corresponding to an initial stage of the treatment plan, one or more intermediate 3D models corresponding to intermediate stages of the treatment plan, and a final 3D model corresponding to a final stage of the treatment plan).

The fabrication computing system 106 may be configured to send the staged 3D models to fabrication equipment 218 for generating, constructing, building, or otherwise producing dental aligners 220. In some embodiments, the fabrication equipment 218 may include a 3D printing system. The 3D printing system may be used to 3D print physical models corresponding the 3D models of the treatment plan. As such, the 3D printing system may be configured to fabricate physical models which represent each stage of the treatment plan. In some implementations, the fabrication equipment 218 may include casting equipment configured to cast, etch, or otherwise generate physical models based on the 3D models of the treatment plan. Where the 3D printing system generates physical models, the fabrication equipment 218 may also include a thermoforming system. The thermoforming system may be configured to thermoform a polymeric material to the physical models, and cut, trim, or otherwise remove excess polymeric material from the physical models to fabricate a dental aligner. In some embodiments, the 3D printing system may be configured to directly fabricate dental aligners 220 (e.g., by 3D printing the dental aligners 220 directly based on the 3D models of the treatment plan). Additional details corresponding to fabricating dental aligners 220 are described in U.S. Provisional Patent Appl. No. 62/522,847, titled “Dental Impression Kit and Methods Therefor,” filed Jun. 21, 2017, and U.S. patent application Ser. No. 16/047,694, titled “Dental Impression Kit and Methods Therefor,” filed Jul. 27, 2018, and U.S. Pat. No. 10,315,353, titled “Systems and Methods for Thermoforming Dental Aligners,” filed Nov. 13, 2018, the contents of each of which are incorporated herein by reference in their entirety.

The fabrication equipment 218 may be configured to generate or otherwise fabricate dental aligners 220 for each stage of the treatment plan. In some instances, each stage may include a plurality of dental aligners 220 (e.g., a plurality of dental aligners 220 for the first stage of the treatment plan, a plurality of dental aligners 220 for the intermediate stage(s) of the treatment plan, a plurality of dental aligners 220 for the final stage of the treatment plan, etc.). Each of the dental aligners 220 may be worn by the patient in a particular sequence for a predetermined duration (e.g., two weeks for a first dental aligner 220 of the first stage, one week for a second dental aligner 220 of the first stage, etc.).

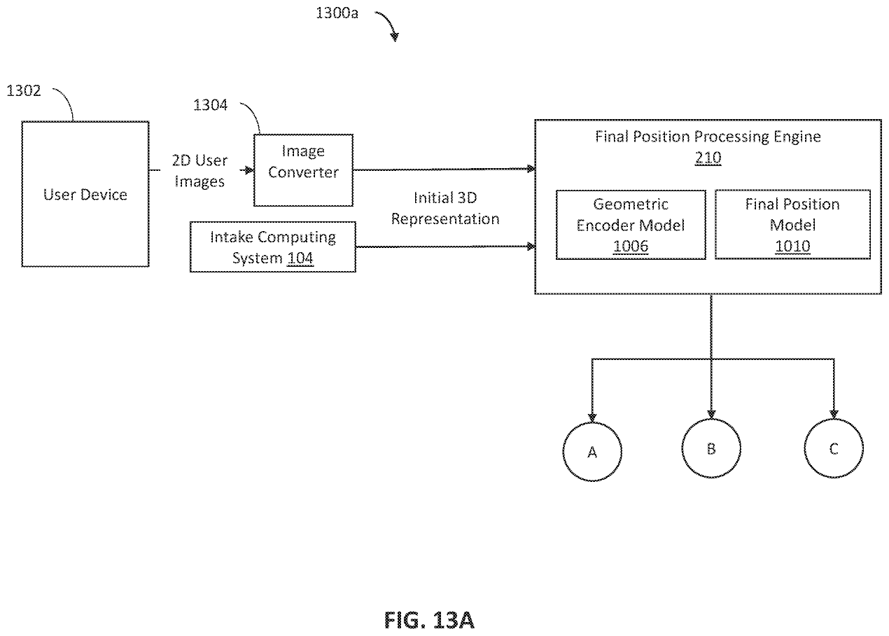

Referring now to FIG. 10, depicted is a view of the final position processing engine 210 of the treatment planning computing system 102 of FIG. 2, according to an illustrative embodiment. As described in greater detail below, the final position processing engine 210 may be configured to automatically determine, derive, or otherwise generate a three-dimensional (3D) representation of a final position of a dentition (also referred to herein as a final 3D representation). The final position processing engine 210 may be configured to generate the final 3D representation responsive to a user selection on the treatment planning terminal 108. The final position processing engine 210 may be configured to generate the final 3D representation by applying an initial 3D representation (e.g., generated by the geometry processing engine 208 as described above) to one or more machine learning models.

The final position processing engine 210 may be configured to receive an initial 3D representation 1002 of a patient's dentition. The final position processing engine 210 may be configured to receive the initial 3D representation 1002 from the geometry processing engine 208. In some embodiments, before the final position processing engine 210 receives initial 3D representation 1002 of the patient's detention, the scan pre-processing engine 202 may normalize and/or standardize the initial 3D representation 1002. For example, the scan pre-processing engine 202 may apply one or more surface smoothing, resampling, and/or artifact removing algorithms to the initial 3D representation 1002. In some embodiments, the initial 3D representation 1002 may be or include a point cloud including points located on surfaces of the patient's dentition. In some embodiments, the initial 3D representation 1002 may be mesh representation, a voxel representation, a spline representation, or any other parametric representation. The initial 3D representation 1002 may include teeth representations 1004 representing each of the teeth (or a group of teeth) in the patient's dentition. Each tooth representation 1004 may include a point cloud including points located on surfaces of the respective tooth. Each of the tooth representations 1004 may together form the initial 3D representation 1002 of the patient's dentition.

The final position processing engine 210 may include, maintain, or otherwise access a geometric encoder model 1006 and, in some cases, a geometric decoder model 1007. The geometric encoder model 1006 may be or include any device, component, or other hardware designed or implemented to convert a tooth representation into a latent space representation (such as a vector), and back into a tooth representation.

The geometric encoder model 1006 may include an encoder. In some embodiments, the geometric encoder model 1006 may be configured to convert, transform, or otherwise generate a vector representation of a 3D representation (such as a point cloud or mesh). The geometric encoder model 1006 may encode and compress the geometry of teeth representations 1004 using one or more deep learning algorithms such as PointNet or PointNet++. The geometric encoder model 1006 may be an encoder of an autoencoder. Accordingly, the geometric encoder model 1006 may be trained using self-supervised learning (or unsupervised learning) based on a loss function between a decompressed/decoded tooth representation prior to compression (e.g., the teeth representation 1004), and a decompressed/decoded tooth representation following compression, encoding the compressed tooth representation into a latent space representation (e.g., vector), and subsequent decompression/decoding into a full point cloud (or other 3D representation).

In the above example, if the geometric encoder model 1006 is an autoencoder, an encoder portion of the autoencoder may learn the latent space representation of one or more teeth in the teeth representation 1004 (e.g., compressing the tooth, encoding the full 3D geometry of each tooth). For example, a convolutional autoencoder may employ convolutional layer(s) and pooling layer(s) to downsample the teeth in the teeth representation 1004 to determine the latent space representation of the teeth in the teeth representation. The convolutional layer(s) convolve the one or more teeth in the teeth representation 1004 with one or more filters to extract features of the teeth representation 1004 to create a feature map. The filters, commonly known as kernels, are of arbitrary sizes and define the field of view for the convolution such that the dimensionality reduces. The pooling layer(s) may further downsample the data by applying a pooling window to the feature map. The pooling layer may be a max pooling layer (or any other type of pooling later) that detects prominent features. In some configurations, the pooling layer may be an average pooling layer. The pooling layer(s) reduce the dimensionality of the feature map to further downsample the feature map.

The latent space representation may include a vector representation of each tooth. The vector representation may be or include a numerical representation of each tooth. The geometric encoder model 1006 may be configured to generate a tensor which combines each vector representation. The tensor may correspond to the number of teeth in a dental arch and the latent space representation for each tooth in the dental arch. For example, a dental arch includes 16 teeth per arch. The geometric encoder model 1006 may be configured to generate a tensor of 16×N, where 16 is a numerical representation of each tooth representation, and N is the number of points in a vector representation of the tooth. Where a dental arch of a patient has a missing tooth, the corresponding tensor generated by the geometric encoder model 1006 may have null or zero values assigned to the corresponding tooth representation in the tensor.

A decoder portion of the autoencoder (e.g., the geometric decoder model 1007) may decompress (or reconstruct) the encoded full 3D geometry of each tooth. The autoencoder operates using the encoder (e.g., geometric encoder model 1006) and decoder (e.g., geometric decoder model 1007) and compares the teeth in the teeth representation 1004 to the decompressed tooth representations following compression, encoding, and subsequent decompression to learn how to better encode the full 3D geometry of each tooth. For example, the target value (e.g., the decompressed tooth representations following compression, encoding, and subsequent decompression) is set to equal the input (e.g., the tooth representation 1004). Accordingly, an example loss function that trains the geometric encoder model 1006 and geometric decoder model 1007 may be based on the 3D error of the reconstructed full 3D geometry of each tooth (or groups of teeth). In some embodiments, the geometric encoder model 1006 may be trained to generate compressed teeth representations of teeth crowns. For example, the target value (e.g., the decompressed tooth representations including the tooth crown following compression, encoding, and subsequent decompression) is set to equal the input (e.g., the tooth representation 1004 including the tooth crown). In some embodiments, the geometric encoder model 1006 may be trained to generate compressed teeth representations of teeth crowns and roots. For example, the target value (e.g., the decompressed tooth representations including the tooth crown and/or roots following compression, encoding, and subsequent decompression) is set to equal the input (e.g., the tooth representation 1004 including the tooth crown and/or roots). The crown or crown and root representations may be similar to the estimated teeth representations described above in connection with the geometry processing engine 208.

The final position processing engine 210 may train the geometric encoder model 1006 and geometric decoder model 1007 until a number of training iterations satisfies a threshold, the error between the decompressed tooth representations following compression, encoding, and subsequent decompression and one or more teeth in the teeth representation 1004 satisfies a threshold, and the like. Training the geometric encoder model 1006 represents the geometric encoder model 1006 encoding the 3D geometry of each tooth into a latent space representation (such as a vector). Accordingly, the compressed teeth representation 1008 (encoded, latent space representation, vector representation) full 3D geometry of each tooth (or a groups of teeth) may be substituted for the full 3D geometry of each tooth (or the groups of teeth) of the teeth representation 1004. In some embodiments, one or more additional vectors may be appended to the compressed teeth representation 1008. For example, the final position processing engine 210 may append a vector including the center of each tooth in 3D coordinates to the compressed teeth representation 1008 output from the geometric encoder model 1006. The final position processing engine 210 may determine the center of each tooth using a global coordinate system, or other coordinate systems.

Once the geometric encoder model 1006 is trained to compress tooth representations as described herein, the geometric encoder model 1006 may be deployed or otherwise used by the final position processing engine 210. Specifically, the final position processing engine 210 may be configured to apply the teeth representations 1004 from an initial 3D representation 1002 to the geometric encoder model 1006 to generate compressed teeth representations 1008. The geometric encoder model 1006 may be configured to generate compressed teeth representations 1008 from the teeth representations 1004 for each of the teeth representations 1004 and/or for a group of teeth.

Once the geometric encoder model 1006 generates a compressed tensor corresponding to the patient's dental arch, (e.g., each of the teeth and/or groups of teeth have been encoded via the geometric encoder model 1006 to compressed teeth representations 1008), the final position processing engine 210 may be configured to apply the tensor to a final position model 1010. The final position model 1010 may be or include any device, component, or other hardware designed or implemented to generate, identify, or otherwise determine tooth movements for each tooth of a dentition from initial positions to final positions. In some embodiments, the final position model 1010 may be configured to determine the final position of the teeth using a trained neural network. The neural network may be trained using supervised learning.

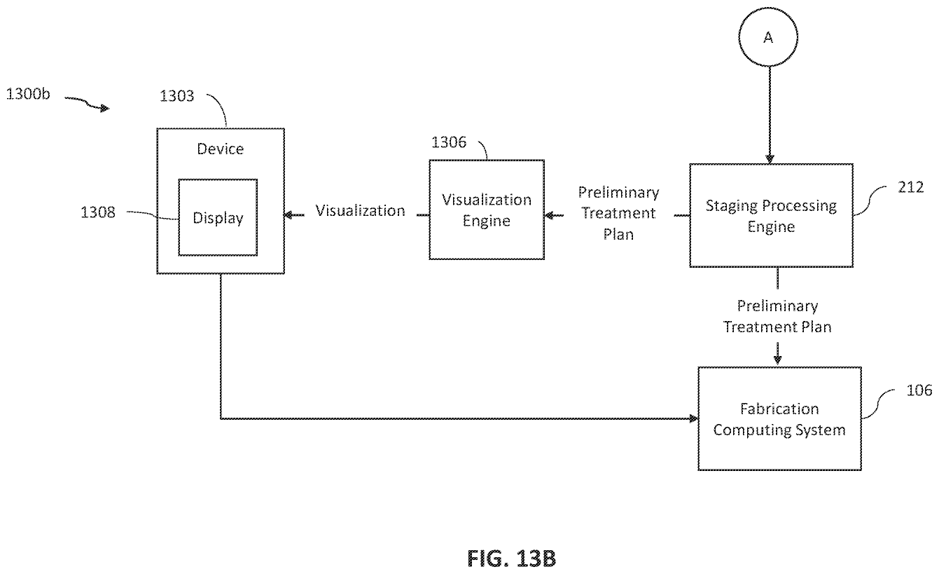

Referring to FIG. 11, a block diagram of an example system 1100 using supervised learning that may be used to determine a movement of the teeth (e.g., the final position of one or more of the patient's teeth post treatment described with respect to the final tooth orientation and translation) is shown according to an example embodiment. Supervised learning is a method of training a machine learning model given input-output pairs. An input-output pair is an input with an associated known output (e.g., an expected output). The final position model 1010 may be trained on known input-output pairs (e.g., full 3D geometric encoded and compressed representations of initial teeth positions and final teeth positions) such that the final position model 1010 can learn how to predict known outputs given known inputs. Once a final position model 1010 has learned how to predict known input-output pairs, the final position model 1010 can operate on unknown inputs to predict an output.

To train the final position model 1010 using supervised learning, training inputs 1102 and actual outputs 1110 may be provided to the final position model 1010. In some embodiments, training inputs 1102 may include a full 3D geometric encoded and compressed representation of each tooth (or a representation of a plurality of teeth) at an initial position. In some embodiments, training inputs 1102 may include encoded projected teeth representation(s) such that each tooth (or groups of teeth) in 3D is converted to an encoded 2D image configured to convey the shape (including the translation and orientation) and location of each tooth (or groups of teeth). Actual outputs 1110 may include a final position of each tooth (or a final position of groups of teeth after the teeth have undergone a treatment plan. The final position of each tooth (or groups of teeth) after the teeth have undergone the treatment plan may be in the form of a 3D representation (e.g., a point cloud) or an encoded latent space representation of each tooth (or groups of teeth).

The inputs 1102 and actual outputs 1110 may be stored in memory or other data structure accessible by the final position processing engine 210. The inputs 1102 and actual outputs 1110 may be received from a historic treatment plan or 3D tooth representation data from a data repository. In some embodiments, the historic treatment plan data may be limited to treatment plans deemed successful (e.g., treatment plans which did not require a mid-course correction, treatment plans receiving positive patient feedback in a survey, etc.). The historic treatment plan data may include, for example, 3D data corresponding to an initial position of the previous patient's dentition, teeth movement data (e.g., movements from the initial position to a respective final position), 3D data corresponding to the final position of the previous patient's dentition (which may be rotation components and translation components obtained from the treatment plan or from a post-treatment impression or intraoral scan), etc.

In an example, the inputs 1102 may be mesh representations of one or more teeth at an initial position. The actual outputs 1110 may be mesh representations of one or more teeth at a final position post treatment. The mesh representations of the teeth at the final position may be received by the final position processing engine 210 by receiving scans of the patient's teeth from the scanning device (e.g., scanning device 214 in FIG. 2) and generating a mesh representation. The mesh representations of the teeth at the final position may also be received by the final position processing engine 210 from a treating dentist (e.g., via a treatment planning terminal 108 in FIG. 1) as a digital representation of an anticipated final position post treatment.

The system 1100 is shown to include a comparator 1108. The comparator 1108 may be configured to compare the transformed predicted output 1109 to the actual output 1110. In some embodiments, the predicted output 1106 may be an encoded latent space representation of teeth at a predicted final position post treatment. For example, the predicted output 1106 may be a M×6 tensor where M represents the number of teeth (e.g., the same number of teeth that was input into the final position model 1010) and 6 represents the orientation and rotation at the final position post treatment (e.g., three translation components and three rotation components). The tensor becomes the rigid body transformation 1111 that, when applied to the initial position of each tooth (e.g., a 3D representation of one or more teeth at an initial position pre-treatment), moves the tooth to a predicted final position post treatment (e.g., the predicted transformed output 1109). In alternate embodiments, the rigid body transformation 1111 may be applied to the initial position of each tooth (e.g., training inputs 1102). In these embodiments, a decoder may decode the transformed decompressed latent space representation into a 3D representation.

The comparator 1108 is configured to compare an error of the 3D representation corresponding to the transformed predicted output 1109 and the 3D representation corresponding to the actual output 1110. In this manner, the loss is determined based on the final treatment plan geometry, not based on the treatment plan movements (e.g., a geometric accuracy assessment, a geometry based final positioning assessment, a geometric feature comparison). Accordingly, center of rotation and other axes are less important compared to the actual final geometry of each tooth (or groups of teeth) because the final geometry of each tooth includes final position rotation and translation components. The comparator 1108 may calculate the loss of the 3D representations using a 3D chamfer distance.

In some embodiments, the comparator 1108 may be configured to compare an actual encoded latent space representation (e.g., actual output 1110) to the predicted compressed transformed output (e.g., predicted transformed output 1109). In these embodiments, the actual encoded latent space representation may be determined by the final position processing engine 210 encoding an actual 3D representation of teeth at a final position post treatment using the trained geometric encoder model 1006. The predicted compressed transformed output may be determined by the final position processing engine 210 applying the rigid body transformation 1111 to the initial position of each tooth (e.g., training inputs 1102). In this manner, the loss is determined based on the final teeth movements (e.g., the translation/rotation components).

During training, the error (represented by error signal 1112) determined by the comparator 1108 may be used to adjust the weights in the final position model 1010 such that the final position model 1010 changes (or learns) over time to generate a relatively accurate prediction of final position of one or more teeth (and/or translation/rotation components of the final position of one or more teeth), using the input-output pairs. The final position model 1010 may be trained using the backpropagation algorithm, for instance. The backpropagation algorithm operates by propagating the error signal 1112. The error signal 1112 may be calculated each iteration (e.g., each pair of training inputs 1102 and associated actual outputs 1110), batch, and/or epoch and propagated through all of the algorithmic weights in the final position model 1010 such that the algorithmic weights adapt based on the amount of error. The error is minimized using a loss function. Non-limiting examples of loss functions may include the square error function, the room mean square error function, and/or the cross entropy error function.

The weighting coefficients of the final position model 1010 may be tuned to reduce the amount of error thereby minimizing the differences between (or otherwise converging) the predicted output 1106 and the actual output 1110. For instance, because the final position model 1010 is being trained to predict the final post treatment teeth position given the initial teeth position, the 3D representation of the predicted final teeth position (e.g., the predicted transformed output 1109) will iteratively converge to the actual final teeth position (e.g., an actual 3D representation of teeth post treatment). The final position processing engine 210 may train the geometric encoder model 1006 until the error determined at the comparator 1108 is within a certain threshold (or a threshold number of batches, epochs, or iterations have been reached). The final position model 1010 and associated weighting coefficients may subsequently be stored in memory or other data repository (e.g., a database) such that the trained final position model 1010 may be employed on unknown data (e.g., not training inputs 1102). Once trained and validated, the final position model 1010 may be employed during testing. During testing, the final position model 1010 may ingest unknown data to predict final teeth positions (e.g., a final 3D representations of one or more teeth, translation/rotation movement components of one or more teeth at a final position after treatment). For example, during testing, the final position model 1010 may ingest tooth position data (in a compressed representation, e.g., compressed teeth representation 1008) to the trained final position model 1010 to predict the final teeth movements (including the translation and rotation components). In a particular example, an upper arch 3D encoded latent space representation of 16×N+3 may be input to the final position model 1010, where 16 represents the 16 individual teeth in an upper arch, N represents the number of points in a vector representation, and 3 represents the position of the center of the tooth in 3D coordinates. The final position model 1010 may output a 16×6 transformation matrix representing each tooth in the upper arch being associated with three translation components and three rotation components of the tooth at a final position after treatment.

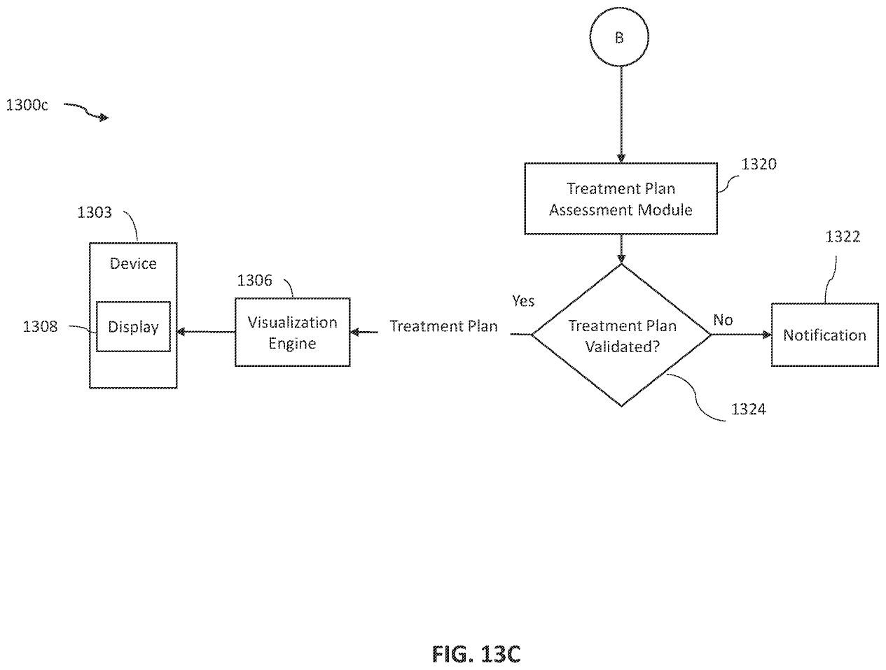

Referring next to FIG. 12, a block diagram of a simplified neural network model 1200 is shown, according to an example embodiment. The neural network may be a machine learning model that is trained to predict a final tooth position. The neural network model 1200 may include a stack of distinct layers (vertically oriented) that transform a variable number of inputs 1202 being ingested by an input layer 1204, into an output 1206 at the output layer 1208.

The neural network model 1200 may include a number of hidden layers 1210 between the input layer 1204 and output layer 1208. Each hidden layer has a respective number of nodes (1212 and 1214). In the neural network model 1200, the first hidden layer 1210-1 has nodes 1212, and the second hidden layer 1210-2 has nodes 1214. The nodes 1212 and 1214 perform a particular computation and are interconnected to the nodes of adjacent layers (e.g., nodes 1212 in the first hidden layer 1210-1 are connected to nodes 1214 in a second hidden layer 1210-2, and nodes 1214 in the second hidden layer 1210-2 are connected to nodes 1216 in the output layer 1208). Each of the nodes (1212, 1214 and 1216) sum up the values from adjacent nodes and apply an activation function, allowing the neural network model 1200 to detect nonlinear patterns in the inputs 1202. Each of the nodes (1212, 1214 and 1216) are interconnected by weights 1220-1, 1220-2, 1220-3, 1220-4, 1220-5, 1220-6 (collectively referred to as weights 1220). Weights 1220 are tuned during training to adjust the strength of the node. The adjustment of the strength of the node facilitates the neural network's ability to predict an accurate output 1206.

In some embodiments, the output 1206 may be one or more numbers (e.g., a matrix of real numbers). The one or more numbers or matrix of real numbers may be representative of tooth movements (e.g., a translation/rotation component associated with a final tooth position after treatment).

Referring back to FIG. 10, and once the final position model 1010 is trained (e.g., via historic treatment plan data), the final position model 1010 may be deployed by the final position processing engine 210 to determine the teeth movements 1012 for the final 3D representation 1016.

It is noted that, while the final position model 1010 and geometric encoder model 1006 are shown as separate models, the final position model 1010 and geometric encoder model 1006 may be sub-components or elements of a single model. For example, a machine learning model may be trained to perform the steps of both the geometric encoder model 1006 and the final position model 1010. In this regard, the final position model 1010 and geometric encoder model 1006 are shown as separate components for purposes of illustration, and the present disclosure is not limited to this particular arrangement. In some embodiments, the machine learning model may include additional or alternative models. For example, the machine learning model may also include a gingiva model configured or trained to determine or learn an evolution of a patient's gingiva during treatment. For example, the gingiva model may be trained on training data including a patient's gingiva at various points in time. The gingiva model may be trained to determine an evolution of the patient's gingiva by predicting the position of gingiva on one or more teeth. The prediction of future gingiva on a patient's teeth may be used for designing or generating aligners which fit a patient's gingiva better.

In some embodiments, the final position processing engine 210 may determine the final 3D representation 1016 by applying the teeth movements 1012 (e.g., the rigid body transformation matrix determined from the final position model 1010) to the teeth representations 1004. In some embodiments, the final position processing engine 210 may determine the final 3D representation 1016 by applying the compressed teeth representations 1008 to the geometric decoder model 1007 to decompress the compressed teeth representations, and generate decompressed teeth representations 1014. In these embodiments, the final position processing engine 210 may apply the teeth movements 1012 (e.g., the rigid body transformation matrix) to the decompressed teeth representations 1014 at the initial position to generate a final 3D representation 1016 (e.g., a 3D teeth representation at a final position after a treatment plan). In some embodiments, the final position processing engine 210 may be configured to apply the rotation/translation components of one or more final tooth positions determined via teeth movements 1012 to the compressed teeth representation 1008. In these embodiments, the geometric decoder model 1007 will decompress the compressed teeth representations at the final post treatment teeth position such that the final 3D representation 1016 is the same as the decompressed teeth representation 1014.

Referring back to FIG. 10 along with FIG. 2 and FIG. 9, and following generating the final 3D representation 1016, the staging processing engine 212 may be configured to determine one or more intermediate 3D representations (or staged 3D models) between the initial 3D representation 1002 and final 3D representation 1016. The intermediate 3D representations may correspond to stages in between the initial position to the final position. For example, the intermediate 3D representation may be represented by applying half of the translation components and half of the rotation components (or some combination of components) to the initial 3D representation 1002. Following generating the intermediate 3D representations (or staged 3D models), the treatment planning computing system 102 may be configured to transmit, send, or otherwise provide the staged 3D models to the fabrication computing system 106 for manufacturing dental aligners 220 as described above.

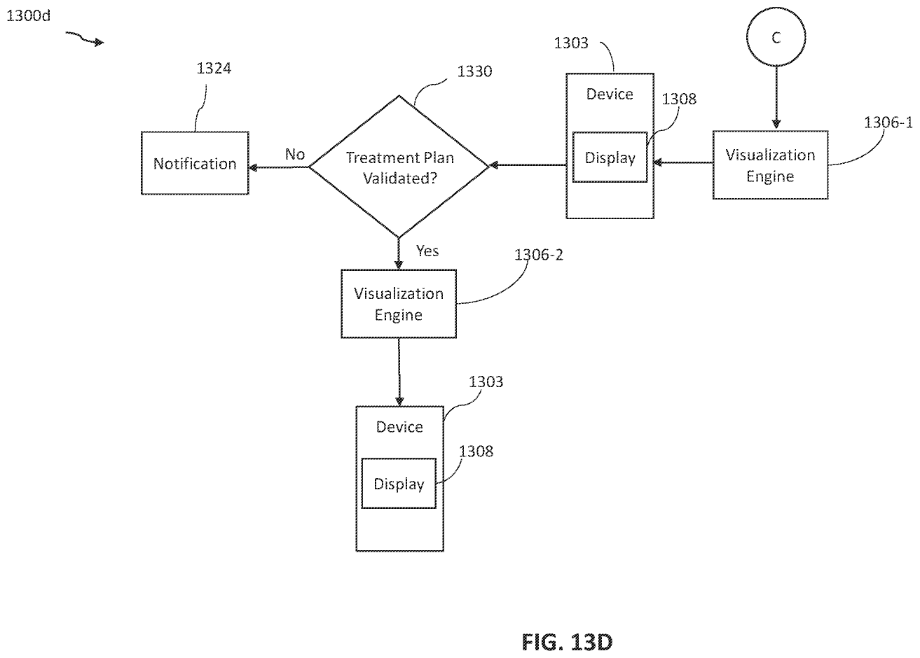

Referring now to FIGS. 13A-D, depicted are systems 1300a-1300d for generating a treatment plan, according to an illustrative embodiment. The systems 1300a-1300d may include components similar to those described above with reference to FIG. 1-FIG. 12. For example, the systems 1300a-1300d may include the intake computing system 104, the final position processing engine 210, the treatment approval terminal 109, the order/purchase terminal 111, the fabrication computing system 106 and/or the staging processing engine 212. The systems 1300a-1300d may also include a user device 1302, an image converter 1304, a visualization engine 1306, a treatment plan assessment module 1320, and/or a display 1308. The systems 1300a-1300d may be used to generate a treatment plan in real-time or near real-time.

The treatment plan may be a preliminary treatment plan similar to the treatment plan generated by the treatment planning computing system 102 described above with reference to FIG. 1-FIG. 12. For example, the preliminary treatment plan may include a preliminary final 3D representation showing a potential final position for a patient along with a series of preliminary intermediate 3D representations showing a progression from an initial position of the patient's teeth to the potential final position. Rather than using the preliminary treatment plan for treating a patient's malocclusion as is typically done with a treatment plan, the preliminary treatment plan may show a possible outcome of a patient should they undergo treatment via dental aligners 220. The treatment plan may also be a final treatment plan. The final treatment plan may be considered the treatment plan that is suitable for orthodontic treatment. That is, the final treatment plan is the treatment plan used to manufacture dental aligners to move the position of a patient's teeth from their initial position to the final after treatment position. The final treatment plan may be the same as the preliminary treatment plan if, for example, the preliminary treatment plan is validated and/or approved.

In some embodiments, a potential patient may capture 2D images of the patient's dentition using a user device 1302. In some embodiments, the potential patient may capture 2D images of a dental impression administered by the patient. The user device may be a smart phone, a camera, etc. The patient may capture a series of 2D images of the patient's dentition from various angles. The user device may upload, send, transmit, or otherwise provide the 2D images to an image converter 1304. The image converter 1304 may be configured to convert the 2D images to an initial 3D representation of the patient's dentition using photogrammetry/triangulation for instance, as discussed with reference to the scan pre-processing engine 202 in FIG. 2. The 3D representation of the patient's detention may be used for generating the treatment plan using the final position processing engine 210. The image converter 1304 may include or use one or more machine learning models, artificial intelligence, or other algorithms for converting 2D images to 3D representations.

In some embodiments, the intake computing system 104 may be configured to generate the initial 3D representation used for generating the treatment plan. For example, the intake computing system 104 may be configured to generate the initial 3D representation from an intraoral scan at an intraoral scanning site as described above with reference to FIG. 2.

The intake computing system 104 (or image converter 1304) may be configured to transmit the initial 3D representation to the final position processing engine 210 to generate a final 3D representation, as described herein. The intake computing system 104 (or image converter 1304) may be configured to transmit the initial 3D representation to the final position processing engine 210 in real-time or near-real time. While not shown, it is noted that, in some embodiments, one or more pre-processing or processing steps may be performed on the initial 3D representation (such as by the scan pre-processing engine 202, the gingival line processing engine 204, segmentation processing engine 206, and/or geometry processing engine 208 as described above with reference to FIG. 2-FIG. 7).

The final position processing engine 210 may be configured to generate a final 3D representation based on the initial 3D representation. The final position processing engine 210 may be configured to generate the final 3D representation using the geometric encoder model 1006 and final position model 1010 as described above with reference to FIG. 10-FIG. 12. In a first embodiment, the final position processing engine 210 may be configured to output the final 3D representation to the staging processing engine 212 to determine whether the final 3D representation is approved/accepted to create a treatment plan (as described in system 1300b in FIG. 13B). In a second embodiment, the final position processing engine 210 may be configured to output the final 3D representation to the treatment plan assessment module 1320 to determine whether the final 3D representation is approved/accepted to create a treatment plan (as described in system 1300c in FIG. 13C). In a third embodiment, the final position processing engine 210 may be configured to output the final 3D representation to the visualization engine 1306 to determine whether the final 3D representation is approved/accepted to create a treatment plan (as described in system 1300d in FIG. 13D).

As shown in FIG. 13B, in system 1300b, the final position processing engine 210 outputs the final 3D representation to the staging processing engine 212. The staging processing engine 212 may be configured to generate a treatment plan using the final 3D representation. The staging processing engine 212 may be configured to generate the treatment plan by generating a plurality of preliminary intermediate 3D representations representing a series of stages from the initial position shown in the initial 3D representation to a preliminary final position shown in the preliminary final 3D representation.

In some embodiments, the final position processing engine 210, the staging processing engine 212, and/or one or more other engines of the system 1300b may perform automated quality control rules or algorithms to ensure that the preliminary final 3D representation and preliminary intermediate 3D representations satisfy one or more rules. For example, the automated quality control rules or algorithms may include ensuring that collisions do not occur at any stage, or any collisions are less than a certain intrusion depth (e.g., less than 0.5 mm). The automated quality control rules or algorithms may include ensuring that certain teeth (such as centrals) are located at approximately a midline of the dentition. The system 1300 may adjust the preliminary final 3D representation and/or preliminary intermediate 3D representation based on an outcome of the automated quality control rules (e.g., to ensure that collisions satisfy the automated quality control rules, to ensure that teeth are located at approximately their intended position, etc.).

The system 1300b is shown to include a visualization engine 1306. The visualization engine 1306 may be or include any device(s), component(s), circuit(s), or other combination of hardware components designed or implemented to determine, produce, or otherwise generate a visualization corresponding to the treatment plan. The visualization engine 1306 may be a component of the treatment planning computing system 102 described above with reference to FIG. 1-FIG. 12. In some embodiments, the visualization engine 1306 may be separate from the treatment planning computing system 102. The visualization generated by the visualization engine 1306 may show a progression from the initial 3D representation (e.g., the teeth at an initial position in a patient's mouth), through the preliminary intermediate 3D representations, and to the final 3D representation (e.g., the teeth at the final position in the patient's mouth). Additionally or alternatively, the visualization may also show the stages of the treatment plan. For example, the visualization engine 1306 may show an initial stage of the treatment plan corresponding to a first 3D model and/or dental aligners corresponding to the initial stage of the treatment plan, one or more intermediate stages of the treatment plan corresponding to one or more intermediate 3D models and/or dental aligners for the intermediate stages of the treatment plan, and a final stage of the treatment plan corresponding to a final 3D model and/or dental aligners for the final stage of the treatment plan. In some embodiments, the visualization engine 1306 may show the final position of teeth after the treatment plan (via a 3D representation).

The visualization engine 1306 may be configured to generate the visualization for rendering on a display 1308 of a device 1303. The visualization engine 1306 may be configured to receive the treatment plan from the staging processing engine 212, and generate the visualization from the treatment plan. The visualization engine 1306 may be configured to generate the visualization as a video, a series of 2D images, or other graphical/visual representation of the treatment plan. The visualization engine 1306 may be configured to transmit, send or otherwise provide the visualization for displaying on a display 1308. In some embodiments, the display 1308 may be a display of the user device 1302. In this regard, the visualization engine 1306 may transmit the visualization back to the user device 1302 (e.g., which uploaded the 2D user images to the image converter 1304) for displaying on the display 1308. The visualization engine 1306 may transmit the visualization to the user device 1302 using an email address or phone number provided by a user when uploading the 2D user images, to a user portal accessible by a user of the user device 1302 via log-in credentials, etc. In some embodiments, the display 1308 may be a display of a computing device at an intraoral scanning site (e.g., an orthodontist office). In this regard, the visualization engine 1306 may be configured to transmit the visualization to a computing device at the intraoral scanning site for displaying on the display 1308. The display 1308 may be located in a room or space in which a user has received an intraoral scan.

The visualization engine 1306 may be configured to transmit the visualization for displaying on the display 1308 in real-time or near real-time. The visualization may therefore show a potential or preliminary visualization of a possible or estimated treatment outcome if the patient (or user) were to be treated via dental aligners 220. As such, the patient may view the visualization on device 1303 (e.g., user device 1302) and determine whether to obtain treatment via dental aligners 220 and/or approve the treatment plan. Similarly, an administrator/technician may view the visualization on device 1303 (e.g., treatment planning terminal 108) and determine whether the dental aligners 220 and/or treatment plan are acceptable (or approved) for treatment.

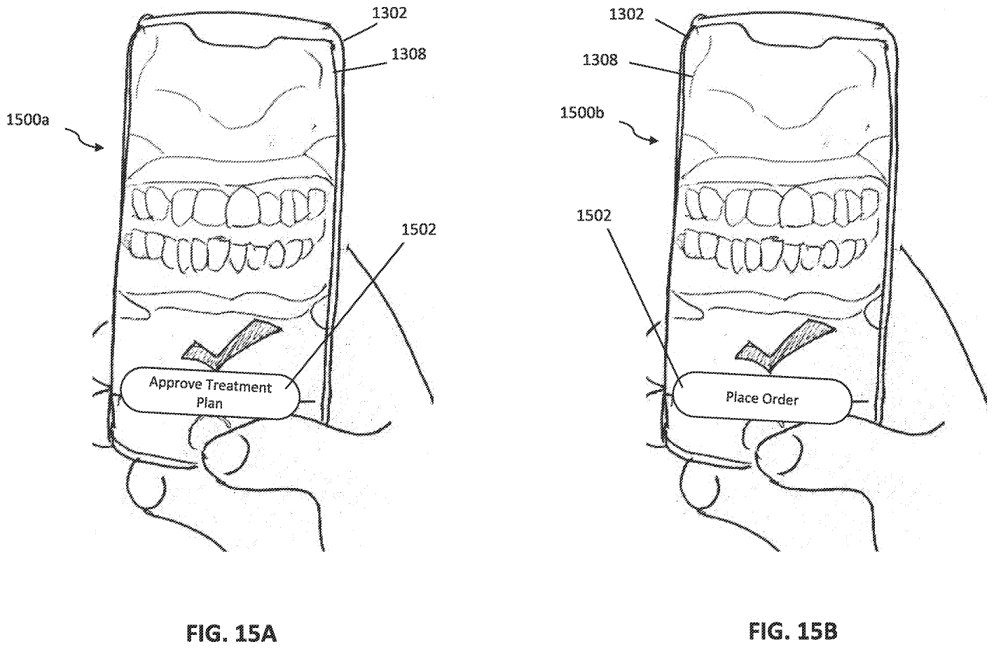

The patient and/or administrator may view the visualization shortly after capturing the 2D user images and/or receiving the intraoral scan. In some embodiments, the treatment plan is a preliminary treatment plan and may not be used for generating the final (e.g., actual) treatment plan for the user. In this regard, the visualization may serve to provide relatively-instantaneous information regarding a potential outcome of treatment, which may assist a user/patient in deciding whether to undergo treatment via dental aligners. In some embodiments, the treatment plan may be used as the final (e.g., actual) treatment plan for the user. For example, the treatment plan may become the final treatment plan after the preliminary treatment plan has been approved by the user (e.g., via the user device 1302) and/or an administrator/technician at the intraoral scanning site via the treatment planning terminal 108). For instance, responsive to the user (and, in some cases, the administrator) approving the treatment plan, the treatment plan may become the final treatment plan and the fabrication computing system 106 may initiate the manufacture/printing/fabrication of the aligners 220. The user may approve/acknowledge the treatment plan by indicating that the user would like to purchase the aligners 220 (e.g., interacting with an “Order Now” button or “Pay Now” button, interacting with a slider, interacting with an object, communicating audibly, communicating with gestures). Referring to FIGS. 15A-15B, depicted are examples of a user approving/acknowledging the treatment plan. As shown, the user device 1302 is displaying a final 3D representation to the user via display 1308. The display 1308 includes interactive button 1502 which indicates that the user approves the final 3D representation (or intends to place an order). In some implementations, the interactive button 1502 may indicate that the user does not approve of the final 3D representation (e.g., the interactive button may communicate “Don't Like”). In yet other implementations, the interactive button 1502 may be a button (or calendar, automated number dialer, and the like) allowing the user to communicate with an office to book an appointment with a treating dentist, technician, orthodontist, administrator, and the like. In other implementations, the display 1308 may communicate multiple interactive buttons that evaluate whether the user accepts/approves of the final 3D representation. In some cases, the user may not be able to order/purchase the dental aligners 220 until an administrator has approved the treatment plan. The user's interaction with interactive button 1502 on the user device 1302 creates an order and/or purchase that is transmitted to an order/purchase terminal (e.g., order/purchase terminal 111 of FIG. 1) for storage/subsequent processing of the order/purchase (e.g., an initiation of a payment/order completion process, as described with reference to FIG. 1).

Referring back to FIG. 13B, in an alternate example, the treatment plan may become the final treatment plan responsive to the final position processing engine 210 satisfying one or more criteria (e.g., a threshold number of users (e.g., technicians, administrators, treating dentists) have approved the treatment plan without modifications, a threshold number of positive reviews regarding the treatment plan from patients). The final position processing engine 210 satisfying the one or more criteria may indicate that the final position processing engine 210 is sufficiently accurate in predicting the final teeth positions.

The system 1300b is also shown to include a fabrication computing system 106. As described herein, the fabrication computing system 106 can include a fabrication computing device and fabrication equipment 218 configured to produce, manufacture, or otherwise fabricate dental aligners 220 corresponding to each stage of the treatment plan where each stage may be representative of a particular 3D model determined by the final 3D representation.

In some embodiments, the fabrication computing system 106 may receive the treatment plan from the staging processing engine 212 and fabricate dental aligners 220 without any user intervention from the user device 1302 and/or from the treatment planning terminal 108 (e.g., a semi-automated or fully automated treatment planning process). Therefore, the treatment plan may be considered the final treatment plan. In some embodiments, the fabrication computing system 106 may be configured to fabricate dental aligners 220 after receiving approval (or acknowledgement) from the user (e.g., via the user device 1302) and/or an administrator/technician at the intraoral scanning site via the treatment planning terminal 108).

As shown in FIG. 13C, the system 1300c describes a fully automated system for verifying the safety and clinical efficacy of an orthodontic treatment plan by performing a clinical assessment on the treatment plan using insights typically obtained and verified during a diagnosis by a professional in the field. The approval/validation of the final tooth position post treatment is automated, as described below. The final position processing engine 210 outputs the final 3D representation to the treatment plan assessment module 1320. The treatment plan assessment module 1320 may include the same or similar components, circuits, hardware, and/or logic as the staging processing engine 212 to convert the 3D representation into a treatment plan. Accordingly, the treatment plan assessment module 1320 may generate the treatment plan (e.g., an initial stage of the treatment plan corresponding to a first 3D model and/or dental aligners for the initial stage of the treatment plan, one or more intermediate stages of the treatment plan corresponding to one or more intermediate 3D models and/or dental aligners for the intermediate stages of the treatment plan, and a final stage of the treatment plan corresponding to a final 3D model and/or dental aligners for the final stage of the treatment plan) and subsequently assess the treatment plan. In some implementations, the final position processing engine 210 outputs the final 3D representation to the staging processing engine 212 to generate the treatment plan and subsequently transmits the generated treatment plan to the treatment plan assessment module 1320. Accordingly, the treatment plan assessment module 1320 may receive one or more stages of the treatment plan. In some implementations, the treatment plan assessment module 1320 may receive one or more stages of the treatment plan and the 3D representation of the teeth.

The treatment plan assessment module 1320 may be any device(s), component(s), circuit(s), or other combination of hardware components designed or implemented to assess the treatment plan. For example, the treatment plan assessment module 1320 may employ rule based logic (e.g., if-then rules) and/or machine learning to assess the treatment plan. For example, a machine learning model may be trained to classify whether a treatment plan is a valid treatment plan given historic treatment plans (e.g., historic treatment plans and corresponding classifications of the validity of the treatment plan). The machine learning model may be a neural network, a random forest, support vector machines, and the like. Additionally or alternatively, the treatment plan assessment module 1320 may obtain one or more metrics from the treatment plan (using one or more engines, as described herein) and compare the metric(s) to criteria. The treatment plan assessment module 1320 assesses the validity of the treatment plan using rule based logic based on the comparison.

In a non-limiting example, the treatment plan assessment module 1320 may identify the positions of the teeth in a received final stage of treatment planning and determine a grading of the treatment plan. If the grading satisfies one or more criteria, the treatment plan assessment module 1320 may determine that the treatment plan is valid.

In another non-limiting example, the treatment plan assessment module 1320 may obtain one or more metrics associated with the treatment plan. The treatment plan assessment module 1320 compares the metrics against one or more criteria. An example of a criterion is whether a smile is aesthetically pleasing. One example metric obtained from the treatment plan and used in the evaluation of whether a smile is aesthetically pleasing may the position of the incisal edges. A smile may be determined to be aesthetically pleasing if the incisal edges are aligned (e.g., the incisal edges may be used by the treatment plan assessment module 1320 to determine the smile line). If the incisal edges are within a predetermined range, the treatment plan assessment module 1320 may determine that the incisal edges are aligned and subsequently that the smile is aesthetically pleasing.

Another metric that may be obtained from the treatment plan by the treatment plan assessment module 1320 may include an evaluation of the occlusion. The treatment plan assessment module 1320 may obtain the occlusion of the individual teeth for the final teeth position (using the treatment plan and/or final 3D representation). If the identified occlusion satisfies one or more criteria, the treatment plan assessment module 1320 may determine that the treatment plan is valid.

The one or more metrics obtained from the treatment plan assessment module 1320 will be used to evaluate whether the treatment plan is a valid treatment plan at 1324 (e.g., whether the treatment plan is a clinically sound and biologically sensible treatment plan). As described herein, the treatment plan assessment module 1320 will determine whether the treatment plan is valid (e.g., at 1324) by comparing the metric(s) to one or more criteria. In the first non-limiting example, the treatment plan is validated if the smile resulting from the treatment plan (e.g., the smile produced by the teeth at the final positions post treatment) is aesthetically pleasing (e.g., the incisal edge alignment satisfies one or more criteria). In the second non-limiting example, the treatment plan is validated if the occlusion is determined to be clinically correct (e.g., satisfying one or more criteria). Accordingly, the treatment plan assessment module 1320 will be used to determine (via the treatment plan validation step 1324) whether the resulting final teeth position, indicated by the 3D representation and/or the final stage, has addressed any previous potential malocclusion of the teeth.

If the treatment plan is validated at 1324, the treatment plan (and/or the 3D representation) may be transmitted to the visualization engine 1306. As described herein, the visualization engine may show the treatment plan (e.g., the final stage), stages of the treatment plan, and/or a 3D representation of the treatment plan (or stages of the treatment plan) to a device 1303 to be displayed via display 1308. The device may be a user device 1302 and/or a treatment approval terminal 109. A user (e.g., treating dentist, administrator, technician, patient) may use the user device 1302 and/or treatment approval terminal 109 to view the displayed and validated treatment plan (e.g., the final stage), stages of the treatment plan, and/or a 3D representation of the treatment plan (or stages of the treatment plan). For example, a patient may use user device 1302 to order the treatment plan, make a payment, approve the treatment plan, initiate the production of one or more dental aligners based on the treatment plan, or some combination. In these embodiments, the patient's order/purchase is transmitted to an order/purchase terminal (e.g., order/purchase terminal 111 of FIG. 1) for storage/subsequent processing of the order/purchase.

If the treatment plan is not validated at 1324, the treatment plan assessment module 1320 may generate a notification 1322 to be transmitted to one or more users (e.g., a patient using user device 1302 and/or a treating dentist using device 1303). The notification may communicate (e.g., using the display of the device 1303, using a microphone of device 1303/1302) that the generated treatment plan was not validated. In one embodiment, the notification may include a reason and/or feedback on why the treatment plan was not validated, and may also include recommendations or requirements to update the treatment plan in such a way that it can be validated and accepted. As an example, such feedback may include a request to move one or more teeth in a way that is different to the initial treatment plan. The treatment plan assessment module 1320 may create the recommendation/feedback using a machine learning model. For example, a machine learning model may be trained to recommend an improvement to a treatment planning model given historic treatment plans (e.g., historic treatment plans and corresponding historic improvements recommended for the treatment plan by a user). In some implementations, the treatment plan assessment module 1320 may prompt a user (e.g., the treating dentist and/or a patient) for additional 2D images of the patient's dentition and/or additional scans of the patient's dentition. In other implementations, the notification 1322 may prompt a user to take additional action(s). For example, a treating dentist may be prompted by the treatment plan assessment module 1320 to edit the treatment plan. Additionally or alternatively, a patient may be prompted to call the treating dentist for additional orthodontic solutions/options. In some implementations, each time a treatment plan is not validated, the treatment plan assessment module 1320 may increment a counter. When the counter reaches and/or exceeds a threshold value, the treatment plan assessment module 1320 may trigger the final position processing engine 210 to retrain the geometric encoder model 1006 and/or the final position model 1010.

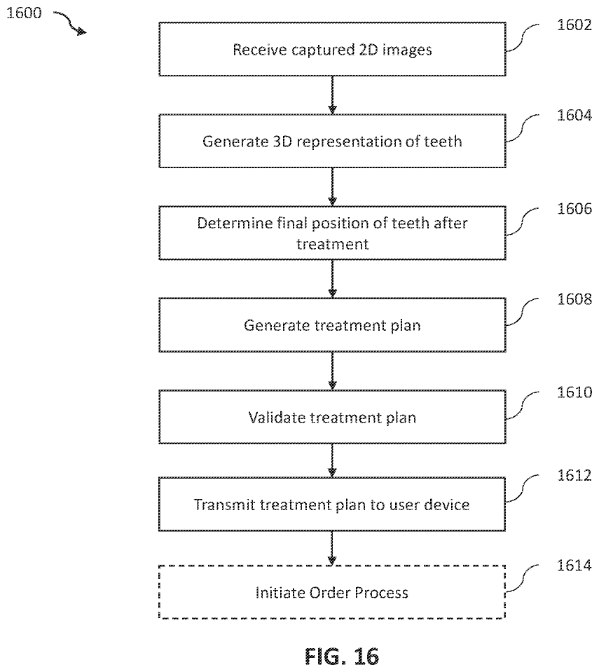

Referring now to FIG. 16, depicted is a flowchart showing a method 1600 of the system 1300c in FIG. 13C. Method 1600 describes automatically verifying the safety and clinical efficacy of the orthodontic treatment plan by performing a clinical assessment on the treatment plan. As a brief overview, at step 1602, one or more processors receive captured 2D images. At step 1604, the processor(s) generate 3D representations of teeth. At step 1606, the processor(s) generate a final position of teeth after treatment. At step 1608, the processor(s) generate a treatment plan. At step 1610, the processor(s) validate the treatment plan. At step 1612, the processor(s) transmit the treatment plan to a user device. At step 1614, the processor(s) initiate an order process. The method 1600 including each of the steps 1602-1614 may be performed by one or more of the devices or components described above with reference to FIG. 1-FIG. 13. Additionally, while shown as being performed in a particular order, it is noted that the steps of the method 1600 may be performed in any order.

At step 1602, one or more processor receive captured 2D images. A patient may capture 2D images of the patient's dentition using a user device 1302. In some embodiments, the patient may capture 2D images of a dental impression administered by the patient. The user device 1302 may be a smart phone, a camera, etc. The patient may capture a series of 2D images of the patient's dentition from various angles. The patient device may upload, send, transmit, or otherwise provide the captured 2D images to the processor(s) such that the processor(s) receive the captured 2D images.

At step 1604, the processor(s) generate 3D representations of teeth. The processor(s) generate 3D representations of teeth using the scan pre-processing engine 202 as described above with respect to FIG. 2. The scan pre-processing engine 202 generates 3D representations of the 2D images using photogrammetry and/or triangulation as described above. Additionally or alternatively, the processor(s) generate 3D representations of the teeth using an image converter.

At step 1606, the processor(s) determine the final position of the teeth after treatment. The processor(s) may determine the final position of the teeth after treatment using the final position processing engine 210 as described above with respect to FIG. 1-FIG. 12. The final position processing engine 210 may feed the 3D representations of teeth at an initial position to geometric encoder model 1006. The geometric encoder model 1006 is an autoencoder and trained using unsupervised based on a loss function between a decompressed/decoded tooth representation prior to compression (e.g., the teeth representation 1004), and a decompressed/decoded tooth representation following compression, encoding the compressed tooth representation into a latent space representation (e.g., vector), and subsequent decompression/decoding into a full point cloud (or other 3D representation). The encoded latent space representation of the one or more teeth may be fed to the final position model 1010 to determine the movement of teeth of a detention from initial positions to final positions and/or a 3D representation of teeth at a final position post treatment. The final position model 1010 may be trained on a training set including a plurality of compressed 3D training representations of dentitions comprising a plurality of teeth, and corresponding tooth movements to respective final positions.

At step 1608, the processor(s) generate a treatment plan. The staging processing engine 212 is configured to generate stages of treatment (e.g., a treatment plan) from the initial position to the final position of the patient's teeth. For example, the staging processing engine 212 generates the stages as 3D digital models of the patient's teeth as the teeth progress from their initial position to their final position, where each 3D digital model may be representative of a particular stage of the treatment plan (e.g., a first 3D model corresponding to an initial stage of the treatment plan, one or more intermediate 3D models corresponding to intermediate stages of the treatment plan, and a final 3D model corresponding to a final stage of the treatment plan).

At step 16010, the processor(s) validate the treatment plan. The treatment plan assessment module 1320 is configured to validate the treatment plan. The treatment plan assessment module 1320 assesses/evaluates the validity of the treatment plan using rule based logic and/or machine learning to classify the validity of the treatment plan.

At step 1612, the processor(s) transmit the validated treatment plan to a user device. A visualization engine 1306 may show the treatment plan (e.g., the final stage), stages of the treatment plan, and/or a 3D representation of the treatment plan (or stages of the treatment plan) to a device 1303 to be displayed via display 1308.

At optional step 1614, the processor(s) may receive an input initiating an order process. The input initiating the order process includes an input from user device 1302 by a patient (or potential patient) to order the treatment plan, make a payment, approve the treatment plan, initiate the production of one or more dental aligners based on the treatment plan, or some combination. The patient's order/purchase is transmitted to an order/purchase terminal (e.g., order/purchase terminal 111 of FIG. 1) for storage/subsequent processing of the order/purchase. The order/purchase terminal 111 communicates prompts to the user device 1302 asking the patient for patient information (e.g., name, physical address, email address, phone number, credit card information) and product information (e.g., quantity, product name) to guide the patient through a payment process.

Referring to FIG. 13D, the system 1300d describes a semi-automatic system for allowing a reviewer to approve a treatment plan. The approval/validation of a final tooth position post treatment is determined using a manual review/approval by a treating dentist as described below. The final position processing engine 210 outputs the final 3D representation to the visualization engine 1306-1.

The visualization engine 1306-1 may include the same or similar components, circuits, hardware, and/or logic as the staging processing engine 212 to convert the 3D representation into a treatment plan. Accordingly, the visualization engine 1306-1 may generate the treatment plan and transmit the treatment plan to device 1303 to be displayed on display 1308. In some implementations, the final position processing engine 210 outputs the final 3D representation to the staging processing engine 212 to generate the treatment plan and subsequently transmits the generated treatment plan to the visualization engine 1306-1. Accordingly, the visualization engine 1306-1 may receive one or more stages of the treatment plan. In some implementations, the visualization engine 1306-1 may receive one or more stages of the treatment plan and the 3D representation of the teeth.

As described herein, the visualization engine 1306-1 may be configured to generate the visualization of the one or more stages of the treatment plan and/or the 3D representation of the teeth as a video, a series of 2D images, or other graphical/visual representation and display such visualization to the display 1308 on device 1303. A technical user (such as a technician, treating dentist, orthodontist, or state licensed professional that has the rights to provide orthodontic treatment to a patient) may review the visualization displayed on display 1308. The technical user may determine whether the treatment plan and/or the 3D representation of the teeth is valid at decision 1330. A treatment plan assessment module (not shown) configured to store the technical user input (e.g., transmitted from a treatment approval terminal 109) may be configured to receive other inputs from the technical user such as improve, request changes, and/or reject the treatment plan and/or the 3D representation of the teeth.

The technical user may input an approval to the treatment plan assessment module if the treatment plan and/or the 3D representation of the teeth are approved by the technical user at 1330. Subsequently, the treatment plan assessment module may transmit and/or apply the treatment plan and/or the 3D representation of the teeth to a visualization engine 1306-2. The visualization engine 1306-2 may convert, transform, reformat or otherwise modify the treatment plan and/or the 3D representation of the teeth such that the treatment plan and/or the 3D representation of the teeth are in a state/format suitable for viewing by a patient/potential patient (e.g., a person considering receiving dental treatment) on device 1303 via display 1308. In some embodiments, after the treatment plan is validated at 1330 by a technical user, the treatment plan may be transmitted to device 1303 (e.g., there may be no visualization engine 1306-2). In some embodiments, the visualization engine 1306-2 may supplement the treatment plan and/or 3D teeth representations visualized by visualization engine 1306-1. For example, the visualization engine 1306-1 may visualize the 3D representation of the teeth to the user device 1303 for review by a technical user. The visualization engine 1306-2 may subsequently visualize the treatment plan, including the initial stage including the 3D digital model of the patient's teeth at their initial position, one or more intermediate stages including 3D digital model(s) of the patient's teeth at one or more intermediate positions, and the final stage including a 3D digital model of the patient's teeth at the final position. Accordingly, the visualization engine 1306-2 may visualize the treatment plan and the 3D teeth representation to be displayed to user device 1302 (e.g., a patient cell phone) via display 1308.

The patient is able to view the 3D teeth representation and/or the treatment plan, in addition to other information relating to the treatment plan (e.g., tooth movements, tooth rotations and translations, clinical indicators, the duration of the treatment plan, the orthodontic appliance that is prescribed to achieve the final tooth position (e.g., aligners), the recommended wear time of the appliance to affect the final tooth position). The information related to the treatment plan may be determined based on historic treatment plans. For example, clinical indicators, the duration of the treatment plan, the orthodontic appliance that is prescribed to achieve the final tooth position (e.g., aligners), and the recommended wear time of the appliance to affect the final tooth position may be determined from a historic treatment plan with a similar initial position and similar final position. Similarly, the information relating to the treatment plan may take into account biomechanical and biological parameters relating to tooth movement, such as the amount and volume of tissue or bone remodeling, the rate of remodeling or the relating rate of tooth movement.

The patient may also order the treatment plan, make a payment, approve the treatment plan, initiate the production of one or more dental aligners based on the treatment plan, book an appointment with a treating dentist, order a product (e.g., an impression kit, aligner) or some combination. In these embodiments, the patient's order/purchase is transmitted to an order/purchase terminal (e.g., order/purchase terminal 111 of FIG. 1) for storage/subsequent processing of the order/purchase.

If the treatment plan is not approved by the technical user at 1330, the technical user will not input an approval to the treatment plan assessment module (not shown) using the treatment approval terminal 109. In response to not receiving an approval (e.g., receiving a denial, receiving no response), the treatment plan assessment module 1320 will generate a notification 1322. The notification 1322 may communicate (e.g., using the display of the device 1308, using a microphone of device 1303) a reminder to the technical user to evaluate the treatment plan and/or a recommendation of an improvement to the treatment plan. The treatment plan assessment module 1320 may create a recommendation of an improvement to the treatment plan using a machine learning model. For example, a machine learning model may be trained to recommend an improvement to a treatment planning model given historic treatment plans (e.g., historic treatment plans and corresponding historic improvements recommended for the treatment plan by a user). In another embodiment, if the treatment plan is not approved by the technical user at 1330, the treatment plan can be routed to be revised or a new treatment plan generated by a user such as a setup technician, an orthodontist or any other person trained to generate a treatment plan. For instance, the treatment plan assessment module 1320 may route the treatment plan to a treatment planning terminal 108.

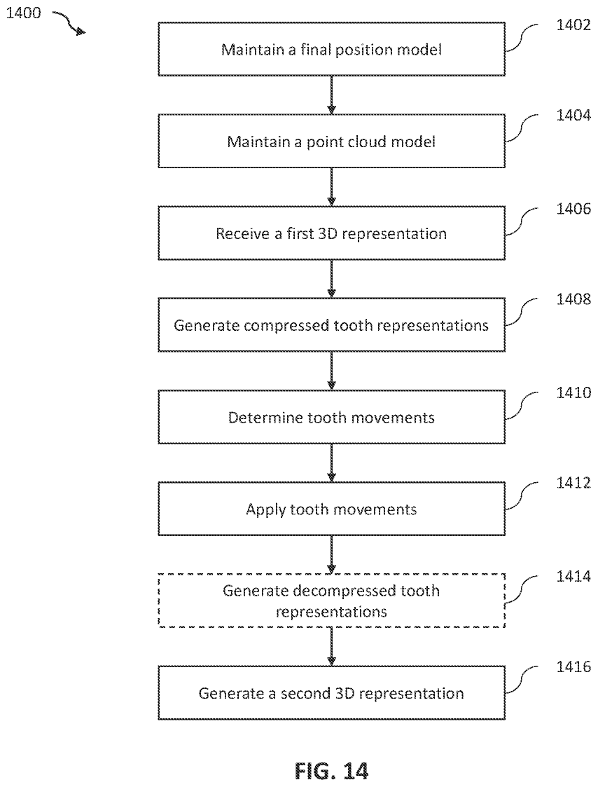

Referring now to FIG. 14, depicted is a flowchart showing a method 1400 of automatically determining a final position of a patient's dentition, according to an illustrative embodiment. As a brief overview, at step 1402, one or more processors maintain a final position model. At step 1404, the processor(s) maintain a geometric encoder model. At step 1406, the processor(s) receive a first three-dimensional (3D) representation. At step 1408, the processor(s) generate compressed tooth representations. At step 1410, the processor(s) determine tooth movements. At step 1412, the processor(s) apply tooth movements. At step 1414, the processor(s) generate decompressed tooth representations. At step 1416, the processor(s) generate a second 3D representation. The method 1400 including each of the steps 1402-1416 may be performed by one or more of the devices or components described above with reference to FIG. 1-FIG. 12. Additionally, while shown as being performed in a particular order, it is noted that the steps of the method 1400 may be performed in any order.

At step 1402, one or more processors maintain a final position model (e.g., final position model 1010). In some embodiments, the processor(s) may maintain a final position model configured to determine movement of teeth of a dentition from initial positions to final positions and/or configured to determine a 3D representation of teeth at a final position post treatment. The processor(s) may be a component or element of the treatment planning computing system 102 described above, such as the final position processing engine 210. The final position model 1010 may be trained on a training set including a plurality of compressed three-dimensional (3D) training representations of dentitions comprising a plurality of teeth, and corresponding tooth movements to respective final positions. In some embodiments, the training set may be limited by previous treatment plans objectively deemed successful. For example, the training set may be limited to treatment plans which did not result in a mid-course correction (e.g., a subsequent treatment plan generated for a patient where the patient's teeth deviated from their intended position at some stage of the treatment plan). The final position model 1010 may be trained as described above with respect to FIG. 11-FIG. 12.

At step 1404, the processor(s) maintain a geometric encoder model 1006. In some instances, the processor(s) may also maintain a geometric decoder model 1007. In some embodiments, the geometric encoder model 1006 is an autoencoder and trained using unsupervised based on a loss function between a decompressed/decoded tooth representation prior to compression (e.g., the teeth representation 1004), and a decompressed/decoded tooth representation following compression, encoding the compressed tooth representation into a latent space representation (e.g., vector), and subsequent decompression/decoding into a full point cloud (or other 3D representation). For example, the target value (e.g., the decompressed tooth representations following compression, encoding, and subsequent decompression) is set to equal the input (e.g., the tooth representation). Accordingly, the loss function that trains the geometric encoder model 1006 is the 3D error of the reconstructed full 3D geometry of each tooth (or groups of teeth).

At step 1406, the processor(s) receive a first three-dimensional (3D) representation. In some embodiments, the processor(s) may receive a first 3D representation of a dentition including a plurality of teeth of a patient in an initial position. The 3D representation may include a plurality of tooth representations including a plurality of points representing surfaces of a respective tooth of the dentition (e.g., a point cloud representation). The first 3D representation may also include a plurality of tooth representations in a mesh representation or voxel (e.g., volumetric pixel) representation, spline representation, or other parametric model representation. In some embodiments, the first 3D representation may be obtained based on a dental impression administered by the patient or an intraoral scan. The first 3D representation may be or include a decompressed, uncompressed, or full 3D representation of the patient's dentition. The first 3D representation (including tooth representations) may include point clouds having points representative of various surfaces of the patient's dentition.

At step 1408, the processor(s) generate compressed tooth representations using the maintained geometric encoder model in step 1404. In some embodiments, the processor(s) may generate a compressed tooth representation for each tooth representation of the first 3D representation. In some embodiments, the processor(s) may generate a compressed tooth representation from groups of teeth of the first 3D representation.

At step 1410, the processor(s) determine tooth movements. In some embodiments, the processor(s) may determine tooth movements (e.g., translation components and rotation components) of the plurality of teeth of the dentition from the initial position to a final position. The processor(s) may determine the tooth movements responsive to applying each compressed tooth representation generated at step 1408 to the final position model 1010.

At step 1412, the processor(s) apply tooth movements. In some embodiments, the processor(s) apply the tooth movements to the compressed tooth representations of the respective teeth of the plurality of teeth, to move the compressed tooth representations into the final position post treatment. In some embodiments, the processor(s) may apply a rigid body transformation including three rotation components and three translation components to the compressed tooth representations using the three translation components and the three rotation components determined at step 1410. In some embodiments, the processor(s) apply the tooth movements to decompressed tooth representations (e.g., following step 1414 described below). In other words, steps 1412 and 1414 may be performed in any particular order. The processor(s) may apply the rigid body transformation to the compressed tooth representations to move the teeth according to the determined tooth movements determined at step 1410. In some embodiments, the processor(s) apply the tooth movements to the first 3D representation (e.g., the first 3D representation received in step 1406). The processor(s) may apply the rigid body transformation to move the teeth into the final post treatment position determined by the final position model 1010.

At step 1414, the processor(s) may generate decompressed tooth representations. For example, the processor(s) may generate decompressed tooth representations if the tooth movements were applied to compressed tooth representations. In some embodiments, the processor(s) may generate decompressed tooth representations using the geometric encoder model 1006. The processor(s) may apply the compressed tooth representations (e.g., determined or generated at step 1408) to a geometric decoder model 1007 to generate the decompressed tooth representations. The geometric decoder model 1007 may be configured to receive the compressed tooth representation and generate decompressed (or full) tooth representations. In some embodiments, the geometric decoder model 1007 may generate the decompressed tooth representations following the determined tooth movements being applied to the compressed tooth representations. In some embodiments, the geometric decoder model 1007 may generate the decompressed tooth representations prior to the determined tooth movements being applied to the compressed tooth representations. In this example, where the geometric decoder model 1007 generates the decompressed tooth representations prior to the determined tooth movements being applied, step 1414 may occur prior to step 1412 described above.

At step 1416, the processor(s) generate a second 3D representation. In some embodiments, the processor(s) may generate a second 3D representation of the dentition comprising the plurality of teeth of the patient in the final position. The processor(s) may generate the second 3D representation using the decompressed tooth representations generated at step 1414. The processor(s) may generate the second 3D representation using the decompressed tooth representations generated at step 1414 with the tooth movements being applied at step 1412. The processor(s) may generate the second 3D representation for rendering on a treatment planning terminal 108. The processor(s) may generate the second 3D representation for generating a treatment plan including stages of the treatment plan.

In some embodiments, the processor(s) may receive an adjustment to the final position of at least one tooth of the plurality of teeth of the dentition. The processor(s) may receive the adjustment from a treatment planning terminal 108. For example, where the second 3D representation is rendered on the treatment planning terminal 108, a user of the treatment planning terminal 108 may provide one or more adjustments to one or more teeth in the second 3D representation. The treatment planning terminal 108 may transmit the adjustment(s) to the processor(s). The processor(s) may update the second 3D representation according to the adjustment received from the treatment planning terminal 108.

In some embodiments, the processor(s) may generate a treatment plan based on the determined tooth movements of the plurality of teeth of the dentition. The processor(s) may generate the treatment plan as described above with reference to FIG. 2. The processor(s) may generate the treatment plan by generating a plurality of intermediate 3D representations of the dentition showing a progression of the plurality of teeth from the initial position to the final position. For example, the staging processing engine 212 may generate the plurality of 3D representations (e.g., the staged 3D models) which show a progression of the teeth from the initial position to the final position. In other words, the plurality of intermediate 3D representations may correspond to a respective stage of the treatment plan. The processor(s) may manufacture (or cause/trigger the manufacturing of) a plurality of dental aligners specific to the dentition and configured to move the plurality of teeth according to the determined tooth movements. For example, the processor(s) may manufacture the dental aligners by transmitting the staged 3D models (or intermediate and final 3D representations) to a fabrication computing system 106. The fabrication computing system 106 may transmit the staged 3D models to fabrication equipment to manufacture the dental aligners.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (e.g., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the Figures. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It is important to note that the construction and arrangement of the systems, apparatuses, and methods shown in the various exemplary embodiments is illustrative only. Additionally, any element disclosed in one embodiment may be incorporated or utilized with any other embodiment disclosed herein. For example, any of the exemplary embodiments described in this application can be incorporated with any of the other exemplary embodiment described in the application. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A method of visualizing a treatment of teeth, the method comprising:
maintaining, by one or more processors, a final position model trained to output movement of teeth of a dentition from initial positions to final positions;
receiving, by the one or more processors from a user device, a first representation of a dentition comprising a plurality of teeth of a user in an initial position, the first representation being based on a two-dimensional (2D) image transmitted from the user device;
determining, by the one or more processors, tooth movements of the plurality of teeth of the dentition from the initial position to a final position using to the final position model;
receiving, by the user device, a graphical visualization of a treatment plan for moving one or more teeth of the user, the graphical visualization generated based on the final position, and the treatment plan being suitable for correcting the user's malocclusion; and
displaying, by the user device, the graphical visualization, wherein the graphical visualization comprises a three-dimensional (3D) representation corresponding to the one or more teeth of the user.

2. The method of claim 1, further comprising generating the treatment plan based on the 2D image transmitted from the user device.

3. The method of claim 2, wherein generating the treatment plan comprises generating, by the one or more processors, a plurality of intermediate 3D representations of the dentition showing a progression of the plurality of teeth from the initial position to the final position, wherein each of the plurality of intermediate 3D representations correspond to a respective stage of the treatment plan.

4. The method of claim 1, wherein the final position model is a trained machine learning model.

5. The method of claim 4, wherein the final position model is trained using an actual 3D representation of one or more teeth at a final position.

6. The method of claim 5, wherein the final position model is trained using a transformed 3D representation of one or more teeth at an initial position.

7. The method of claim 1, further comprising receiving, based on a user interaction with the graphical visualization, a user approval of the treatment plan.

8. The method of claim 7, wherein the user approval comprises one or more of receiving an order for dental aligners fabricated based on the treatment plan, receiving a request for an impression kit, receiving a booking of an appointment for an intraoral scan, receiving a request for an appointment for an intraoral scan, receiving a payment from the user, or the user device causing the treatment plan to be shared with another user.

9. The method of claim 7, wherein the user approval comprises a user input received on the user device, the user input comprising at least one of an interaction with a button, an interaction with a slider, an interaction with an object, an audible communication, or a gesture communication.

10. The method of claim 7, further comprising displaying, by the user device, a series of prompts to guide the user through an order completion process.

11. A method of visualizing a treatment of teeth, the method comprising:
maintaining, by one or more processors, a final position model trained to output movement of teeth of a dentition from initial positions to final positions;
receiving, by the one or more processors from a user device, a first representation of a dentition comprising a plurality of teeth of a user in an initial position, the first representation being based on a two-dimensional (2D) image transmitted from the user device;
determining, by the one or more processors, tooth movements of the plurality of teeth of the dentition from the initial position to a final position using to the final position model; and
providing, by the one or more processors to the user device, a graphical visualization of a treatment plan for moving one or more teeth of the user, the graphical visualization generated based on the final position, and the treatment plan being suitable for correcting the user's malocclusion;
wherein providing the graphical visualization to the user device causes the user device to display the graphical visualization, wherein the graphical visualization comprises a three-dimensional (3D) representation corresponding to the one or more teeth of the user.

12. The method of claim 11, further comprising generating the treatment plan based on the 2D image transmitted from the user device.

13. The method of claim 12, wherein generating the treatment plan comprises generating, by the one or more processors, a plurality of intermediate 3D representations of the dentition showing a progression of the plurality of teeth from the initial position to the final position, wherein each of the plurality of intermediate 3D representations correspond to a respective stage of the treatment plan.

14. The method of claim 11, wherein the final position model is a trained machine learning model.

15. The method of claim 14, wherein the final position model is trained using an actual 3D representation of one or more teeth at a final position.

16. The method of claim 15, wherein the final position model is trained using a transformed 3D representation of one or more teeth at an initial position.

17. The method of claim 11, further comprising receiving, based on a user interaction with the graphical visualization, a user approval of the treatment plan.

18. The method of claim 17, wherein the user approval comprises one or more of receiving an order for dental aligners fabricated based on the treatment plan, receiving a request for an impression kit, receiving a booking of an appointment for an intraoral scan, receiving a request for an appointment for an intraoral scan, receiving a payment from the user, or the user device causing the treatment plan to be shared with another user.

19. The method of claim 17, wherein the user approval comprises a user input received on the user device, the user input comprising at least one of an interaction with a button, an interaction with a slider, an interaction with an object, an audible communication, or a gesture communication.

20. The method of claim 17, further comprising providing, by the one or more processors to the user device, a series of prompts to guide the user through an order completion process.

21. A treatment planning system comprising:
one or more processors; and
memory storing instructions that, when executed by the one or more processors, cause the one or more processors to:
maintain a final position model trained to output movement of teeth of a dentition from initial positions to final positions;
receive, from a user device, a first representation of a dentition comprising a plurality of teeth of a user in an initial position, the first representation being based on a two-dimensional (2D) image transmitted from the user device;
determine tooth movements of the plurality of teeth of the dentition from the initial position to a final position using to the final position model; and
provide, to the user device, a graphical visualization of a treatment plan for moving one or more teeth of the user, the graphical visualization generated based on the final position, and the treatment plan being suitable for correcting the user's malocclusion;
wherein providing the graphical visualization to the user device causes the user device to display the graphical visualization, wherein the graphical visualization comprises a three-dimensional (3D) representation corresponding to the one or more teeth of the user.

22. The treatment planning system of claim 21, wherein the memory further stores instructions that, when executed by the one or more processors, cause the one or more processors to generate the treatment plan based on the 2D image transmitted from the user device.

23. The treatment planning system of claim 22, wherein generating the treatment plan comprises generating a plurality of intermediate 3D representations of the dentition showing a progression of the plurality of teeth from the initial position to the final position, wherein each of the plurality of intermediate 3D representations correspond to a respective stage of the treatment plan.

24. The treatment planning system of claim 21, wherein the final position model is a trained machine learning model.

25. The treatment planning system of claim 24, wherein the final position model is trained using an actual 3D representation of one or more teeth at a final position.

26. The treatment planning system of claim 25, wherein the final position model is trained using a transformed 3D representation of one or more teeth at an initial position.

27. The treatment planning system of claim 21, wherein the memory further stores instructions that, when executed by the one or more processors, cause the one or more processors to receive, based on a user interaction with the graphical visualization, a user approval of the treatment plan.

28. The treatment planning system of claim 27, wherein the user approval comprises one or more of receiving an order for dental aligners fabricated based on the treatment plan, receiving a request for an impression kit, receiving a booking of an appointment for an intraoral scan, receiving a request for an appointment for an intraoral scan, receiving a payment from the user, or the user device causing the treatment plan to be shared with another user.

29. The treatment planning system of claim 27, wherein the user approval comprises a user input received on the user device, the user input comprising at least one of an interaction with a button, an interaction with a slider, an interaction with an object, an audible communication, or a gesture communication.

30. The treatment planning system of claim 27, wherein the memory further stores instructions that, when executed by the one or more processors, cause the one or more processors to provide, to the user device, a series of prompts to guide the user through an order completion process.
